(19) United States
(12) Patent Application Publication
(10) Pub. No.: US 2025/0312412 A1
(43) Pub. Date: Oct. 9, 2025

(54) METHODS FOR TREATING DRY EYE DISEASE

(71) Applicants: The Schepens Eye Research Institute, Inc., Boston, MA (US); Aramis Biosciences, Inc., Medfield, MA (US)

(72) Inventors: Sunil Chauhan, Boston, MA (US); Reza Dana, Boston, MA (US); David C. Lacey, Medfield, MA (US); David S. Tierney, Medfield, MA (US)

(21) Appl. No.: 18/872,589

(22) PCT Filed: Jun. 7, 2023
(86) PCT No.: PCT/US2023/068089

§ 371 Date: Dec. 6, 2024

Related U.S. Application Data
(60) Provisional application No. 63/350,365, filed on Jun. 8, 2022.

(51) Int. Cl. A61K 38/16; A61K 9/00; A61K 31/015; A61K 31/404; A61K 38/10; A61P 27/04; C07K 16/24; C07K 16/28

(52) U.S. Cl. CPC A61K 38/16; A61K 9/0048; A61K 31/015; A61K 31/404; A61K 38/10; A61P 27/04; C07K 16/244; C07K 16/2866

(57) ABSTRACT

Methods to treat dry eye disease and to slow, inhibit or delay progression of dry eye disease in a subject are provided. The methods comprise administering a composition comprising a therapeutically effective amount of a compound that inhibits binding of an inflammatory interleukin-17 (IL-17) cytokine to an IL-17 receptor. In embodiments, the compounds is a protein or a peptide.

### REFERENCE TO SEQUENCE LISTING, TABLE OR COMPUTER PROGRAM

This application contains a Sequence Listing which has been submitted electronically in XML format. The Sequence Listing XML is incorporated herein by reference. The XML file, created on 7 Jun. 2023, is named 113665-0038-8001WO00_SEQ.xml and is 186,910 bytes.

### TECHNICAL FIELD

The subject matter described herein relates to methods to treat dry eye disease and to slow, inhibit or delay progression of dry eye disease in a subject by administering a composition comprising a therapeutically effective amount of a compound that inhibits binding of an inflammatory interleukin-17 (IL-17) cytokine to an IL-17 receptor.

### BACKGROUND

Dry eye is a multifactorial, inflammatory, immune-mediated disease of the ocular surface that results in symptoms of discomfort, visual disturbance, and tear film instability as well as epithelial cell damage at the ocular surface. The normal tear film is a relatively stable, thin film composed of a superficial lipid layer and an aqueous layer intermixed with a mucus gel layer which is partially adherent to the corneal and conjunctival surface epithelium. Natural tear film is important for the lubrication and maintenance of the refractive surface of the eye. Dry eye disease is a complex disorder characterized by a dysfunction of one or more components of the tear film, leading to the loss of tear film homeostasis or stability, a hyperosmotic shift in the tear film osmotic balance, and/or an inadequate amount of fluid on the ocular surface. This is characterized by rapid break-up of the tear film and numerous symptoms, including eye dryness, ocular pain, burning/stinging, foreign body sensation, itching, and photophobia.

### BRIEF SUMMARY

The following aspects and embodiments thereof described and illustrated below are meant to be exemplary and illustrative, not limiting in scope.

In one aspect, a method to treat dry eye disease in a subject diagnosed with dry eye disease is provided. The method comprises administering directly to the eye of a subject in need a composition comprising a therapeutically effective amount of a compound that inhibits binding of an inflammatory interleukin-17 (IL-17) cytokine to an IL-17 receptor.

In another aspect, a method to slow, inhibit or delay progression of dry eye disease in a subject is provided. The method comprises administering directly to the eye of a subject in need a composition comprising a therapeutically effective amount of a compound that inhibits binding of an inflammatory interleukin-17 (IL-17) cytokine to an IL-17 receptor.

In another aspect, a method to reverse progression of dry eye disease in a subject is provided. The method comprises administering directly to the eye of a subject in need a composition comprising a therapeutically effective amount of a compound that inhibits binding of an inflammatory interleukin-17 (IL-17) cytokine to an IL-17 receptor.

In another aspect, a method to restore tear film homeostasis on the surface of an eye is provided. The method comprises administering directly to the eye of a subject in need a composition comprising a therapeutically effective amount of a compound that inhibits binding of an inflammatory interleukin-17 (IL-17) cytokine to an IL-17 receptor.

In one embodiment, the method comprises identifying a subject with mild or moderate dry eye disease.

In one embodiment, the method comprises identifying a subject with mild, moderate or severe dry eye disease.

In one embodiment, identifying a subject comprises use of a questionnaire.

In one embodiment, the questionnaire is selected from the Visual Analog Scale (VAS), the Ocular Surface Disease Index (OSDI) questionnaire, the Symptom Assessment Questionnaire iN Dry Eye (SANDE) questionnaire, Dry Eye Questionnaire (DEQ), and the Standard Patient Evaluation of Eye Dryness Questionnaire (SPEED).

In one embodiment, the method comprises identifying a subject with mild or moderate dry eye disease and monitoring progression of dry eye disease.

In an embodiment, progression of dry eye disease is monitored using an eye dryness visual analog scale, for example, VAS or SANDE.

In one embodiment, the composition administered comprises a compound selected from a biological molecule or an organic synthetic compound.

In one embodiment, the organic, synthetic compound is a spirocyclic indane compound or a spirocyclic oxoindoline compound.

In one embodiment, the biological compound is an antibody with binding affinity for IL-17 cytokine or an antibody with binding affinity for an IL-17 receptor.

In one embodiment, the antibody is a monoclonal antibody, a polyclonal antibody, a single-chain antibody, a humanized, recombinant antibody, a chimeric antibody, or an antibody fragment.

In one embodiment, the antibody is selected from the group consisting of afasevikumab, bimekizumab, brodalumab, ixekizumab, izokibep netakimab, perakizumab, secukinumab, sonelokimab, tibulizumab, vunakizumab, ABY-035, CJM-112, CNTO-6785, DC-806/5-011806, FPP-003, GR-1501, HB-0017, IMU-035, LZM-012, QX-002N, BH-1657, HB-0043, HT-0017, ILCT-1001, IQ-001, LEO 153339/LP0200, LP-0200, MT-6194, MYMD-1, ND-016, SCT-650A, SM-17, YBL-004, ABM-60, ETI-1023, LQ-025, LQ-026, ABBV-257, AFB-035, ANB-004, BCD-121, COVA-322, CYT-017-IL17Qb, DLX-2882, DLC-2907, DLX-2909, DLX-3003, E-34935, E-35018, E-35762, E-36041, EBI-006, HEISCO-II-002, IL-17-RC, MEDI-571, MOR-106, MP-0230, PRS-190, SCH-900117, Y-320, ABT-122, BITS-7201A, CDP-435, EBI-028, IL-17E, JNJ-6118104, KHK-4827, and RG 7624.

In one embodiment, the biological compound is a protein or a peptide that specifically inhibits IL-17A binding to interleukin 17A receptor.

In one embodiment, the peptide consists of a contiguous sequence of between 10-25, 12-20, 13-18, 13-16, 14-16, or 15 amino acid residues, the contiguous sequence having at least about 70%, 80%, 85%, 90%, 95%, 96%, 97%, 98% or 99% sequence identity to the sequences of Formula I. In another embodiment, the peptide consists of a contiguous sequence of between 12-18 amino acid residues, the contiguous sequence having at least about 70% sequence identity to SEQ ID NO: 1. In another embodiment, the peptide consists of a contiguous sequence of between 12-18 amino acid residues, the contiguous sequence having at least about 70% sequence identity to any one of the sequences identified herein as SEQ ID NOs: 1-166. In another embodiment, the peptide consists of a contiguous sequence of between 12-18 amino acid residues, the contiguous sequence having at least about 70% sequence identity to any one of the sequences identified herein as SEQ ID NOs: 1-214. In another embodiment, the peptide consists of a contiguous sequence of between 12-18 amino acid residues, the contiguous sequence having at least about 70% sequence identity to any one of the sequences identified herein as SEQ ID NOs: 1-166, provided that the peptide is not SEQ ID NO: 167-214.

In one embodiment, the peptide comprises an amino acid sequence of Formula I:

X₁-X₂-X₃-X₄X₅-X₆-X₇-X₈-X₉-X₁₀-X₁₁-X₁₂-X₁₃-X₁₄-X₁₅ Formula (I),

wherein
X₁ is I, V or L;
X₂ is H, M, R, K or E;
X₃ is V, F or I;
X₄ is T, Q, S, Y or N;
X₅ is I, F or V;
X₆ is P or G;
X₇ is A, Q, or L;
X₈ is D, E, or Q;
X₉ is L, W, F, V or I;
X₁₀ is W, Y or F;
X₁₁ is D, E or N;
X₁₂ is W or F;
X₁₃ is I, V, F or L;
X₁₄ is N, R, Q or E; and
X₁₅ is K, R, H or E,
In an embodiment, the sequence is not IHVTIPADLWDWINK (SEQ ID NO: 167).

In one embodiment, the amino acid sequence of Formula I is wherein
(a) X₁ is I or V,
   X₂ is H, M or R,
   X₃ is V or F;
   X₄ is T or Q;
   X₅ is I, F or V;
   X₆ is P or G;
   X₇ is A or Q;
   X₈ is D or E;
   X₉ is L;
   X₁₀ is W or Y;
   X is D or E;
   X₁₂ is W;
   X₁₃ is I or V;
   X₁₄ is N, R or E; and
   X₁₅ is K, R or E;
or
(b) X₁ is I or V;
   X₂ is H or M,
   X₃ is V;
   X₄ is T;
   X₅ is I;
   X₆ is P;
   X₇ is A;
   X₈ is D;
   X₉ is L, W, F, V or I;
   X₁₀ is W or Y;
   X₁₁ is D or E;
   X₁₂ is W;
   X₁₃ is I or V;
   X₁₄ is N, R or E; and
   X₁₅ is K, R or E.

In an embodiment, the sequence is not IHVTIPADLWDWINK (SEQ ID NO: 167), HVTIPADLWDWIN (SEQ ID NO: 168), IHVTIPADLWDWI (SEQ ID NO: 169) or IHVTIPADLWDW (SEQ ID NO: 170).

In one embodiment, the peptide bound its C- and/or N-terminal to a protective cap group, wherein the protective cap group bound to a C-terminal is selected from the group consisting of amides, aldehydes, esters, p-nitroanilide, 7-amino-4-methylcoumarin. In one embodiment, the protective group cap bound to a N-terminal is selected from the group consisting of acetyl, formyl, pyroglutamyl, fatty acids, urea, carbamate sulfonamide, and alkylamine.

In one embodiment, the peptide in the form of a dimer formed by two peptides, wherein each peptide is a peptide of Formula I.

In one embodiment, the two peptides in the dimer are linked by a polyethylene spacer.

In one embodiment, the peptide is in the form of a bioconjugate comprising the peptide of Formula I and a biomolecule, wherein the biomolecule is bound to the N- and/or C-terminal of the peptide.

In one embodiment, the biomolecule is selected from the group consisting of capric acid, capronic acid, ascorbic acid, NAG-NAM, NAG, NAM, hyaluronic acid, alginic acid, chitin, (GalNAc)2, Gal-alpha1,3-GalNAc and trigalacturonic acid.

In one embodiment, the peptide is in a pharmaceutically acceptable composition that comprises at least one pharmaceutically acceptable excipient.

In one embodiment, administering comprises instilling onto the ocular surface.

In one embodiment, administering comprises instilling into the conjunctival sac.

In one embodiment, administering comprises once daily administering.

In one embodiment, the therapeutically effective dose of the peptide is between about 0.01-1,000 μM or between about 0.05-750 μM or between about 0.05-500 μM, or is at least about 500 μM.

In one embodiment, the dose is administered once daily.

In one embodiment, administering is for a period of at least about 3 weeks, wherein the signs and/or symptoms of dry eye disease are resolved for a disease free period in the third week of said administering. In another embodiment, administering is for a period of at least about 3 weeks, wherein the signs of dry eye disease are resolved for a disease free period in the third week of said administering.

In another aspect, a method to restore goblet cell function and/or density in an eye with signs and/or symptoms of dry eye disease or a method of preventing goblet cell loss in the eye of a person with dry eye disease is provided. The method comprises topically administering to the eye of a subject in need, a therapeutically effective amount of a compound that inhibits IL-17A binding to interleukin 17A receptor.

In another aspect, a method to improve balance of function of the lacrimal gland, the Meibomian glands and conjunctival goblet cells to restore tear film homeostasis in a subject is provided. The method comprises topically administering to the eye of a subject in need, a therapeutically effective amount of a compound that inhibits IL-17A binding to interleukin 17A receptor.

In another aspect, a method to increase basal tear production in a human subject is provided. The method comprises topically administering to the eye of a subject in need, a therapeutically effective amount of a compound that inhibits IL-17A binding to interleukin 17A receptor.

In one embodiment, the subject is a subject at least about 50 years of age (an “elderly” subject). In an embodiment, the elderly subject has hormonal changes and/or is over age 50, 55 or 60.

In one embodiment, the compound for use in any of the methods is not any one or more of DLSAVCWAFPWDPECH (SEQ ID NO: 171), DSSAVCWAFPHHPLCHMKAT (SEQ ID NO: 172), ADADMCWFFPTSPWCH (SEQ ID NO: 173), DSSAVCWAFPYLPECH (SEQ ID NO: 174), DISAVCWAFPFDPECH (SEQ ID NO: 175), AYECPRLEYDMFGALHCLPS (SEQ ID NO: 176), CPRLEYDMFGALHCL (SEQ ID NO: 177), CLDLQYDPWGALHCI (SEQ ID NO: 178), CFDLQYDPWGALHCI (SEQ ID NO: 179), CLDLQYDMFGALHCV (SEQ ID NO: 180), CLDLVYDPWGALHCI (SEQ ID NO: 181), CWVLEYDMFGALHCR (SEQ ID NO: 182), CWALEYDMFGYLHCR (SEQ ID NO: 183), CWVLEYDMFGFLHCR (SEQ ID NO: 184), CWVLEYDMFGYLHCR (SEQ ID NO: 185), and GPYYFDSSGYLYYYYGLDV (SEQ ID NO: 186).

In addition to the exemplary aspects and embodiments described above, further aspects and embodiments will become apparent by reference to the drawings and by study of the following descriptions.

Additional embodiments of the present methods, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIG. 1 is a graph of corneal fluorescein staining (CFS) scored using the National Eye Institute Industry Workshop Scale of 0-15, as a function of time, in days, in mice induced with dry eye disease by placing them in a controlled-environment chamber (relative humidity: <20%, dry airflow: 15 L/min, temperature: 21˜23° C.) for 14 days. Corneal epithelial disease was evaluated using corneal fluorescein staining (CFS) and scored using the National Eye Institute Industry Workshop Scale of 0-15, at day 0 (normal baseline), day 4 (before instillation of topical therapy), day 7, day 10, and day 14 in mice treated by topical application to the eye with an anti-IL-17A antibody once daily (closed circles) or twice daily (closed squares), lifitegrast (XIIDRA®) twice daily (inverted triangles), cyclosporine (RESTASIS®) twice daily (triangles), or saline twice daily (diamonds).

FIGS. 2A-2B are bar graphs showing the effect of the indicated treatments on improving the immunoregulatory function of Treg cells, as measured by FoxP3 expression, (FIG. 2A) and suppressing the differentiation of naïve T cells into pathogenic Th17 cells (FIG. 2B) in the lymph nodes of mice from the study in FIG. 1 and Example 1.

FIGS. 3A-3E are images of conjunctival tissue showing frequencies of goblet cells (large black spots in top cell layer) from healthy mice with no dry eye disease (FIG. 3A) and from mice induced with dry eye disease by placing them in a controlled-environment chamber and treated twice daily with saline as a control (FIG. 3B), cyclosporine ophthalmic emulsion (RESTASIS®; FIG. 3C), lifitegrast ophthalmic solution (XIIDRA®) (FIG. 3D), or anti-IL-17A antibody (FIG. 3E).

FIG. 3F is a bar graph of the percent of goblet cells for normal (healthy) mice and for mice induced with dry eye disease by placing them in a controlled-environment chamber and treated with saline, cyclosporine, lifitegrast or an IL-17A inhibitor.

FIGS. 4A-4C are images of microscopic inspection of human corneal epithelial cells (FIG. 4A) and of human corneal epithelial cells in co-culture with Th17 cells treated with saline vehicle (FIG. 4B) or with an IL-17 inhibitor, an IL-17A antibody (FIG. 4C).

FIGS. 5A-5F are images from bright-light microscopic inspection (40×) of human corneal epithelial cells (FIG. 5A) and of human corneal epithelial cells in co-culture with Th17 cells treated with saline vehicle (FIG. 5B) or with an IL-17 inhibitor (SEQ ID NO: 1 (VHVTIPADLWDWINK)) at doses of 0.1 μM (FIG. 5C), 1 μM (FIG. 5D), 10 μM (FIG. 5E), and 100 μM (FIG. 5F).

FIG. 6A is a graph of corneal fluorescein staining (CFS) scored using the National Eye Institute Industry Workshop Scale of 0-15 at day 0 (normal baseline), day 4 (before instillation of topical treatments) and during treatment an IL-17A antibody (inverted triangles) at a dose of 1% of an IL-17 inhibitor, or an IL-17 inhibitor (SEQ ID NO: 1 (VHVTIPADLWDWINK)), at doses of either 0.05 wt % (circles) or 0.1 wt % (diamonds) in phosphate-buffered saline, administered twice daily on days 4-12; the control group was administered saline vehicles (squares) twice daily on days 4-12.

FIG. 6B is a bar graph of the percent of Th17 cells in the draining lymph nodes of the same mice from FIG. 6A, including mice with no dry eye disease and mice induced with dry eye disease by placing them in a controlled-environment chamber and treated with saline (control) or with an IL-17A antibody at a dose of 1 wt % (10 mg/mL) or an IL-17 inhibitor (SEQ ID NO: 1 (VHVTIPADLWDWINK)), at a dose of either 0.05 wt % or 0.1 wt %, in phosphate-buffered saline, administered twice daily for 9 days.

FIG. 7 is a graph of corneal fluorescein staining (CFS) scored using the National Eye Institute Industry Workshop Scale of 0-15 as a function of time, in days, during a treatment phase of a study on mice with dry eye disease and treated with saline vehicle twice daily (open squares) or with an IL-17 inhibitor (SEQ ID NO: 1 (VHVTIPADLWDWINK)) formulated in phosphate-buffered saline at concentrations of 50 μM (administered twice daily, inverted triangles), 250 μM (administered to separate groups once daily (diamonds) and twice daily (solid squares)), and 500 μM (administered once daily, circles).

FIG. 8 is a graph of corneal fluorescein staining (CFS) scored using the National Eye Institute Industry Workshop Scale of 0-15 as a function of time, in days, in three groups of mice after induction of dry eye disease and during treatment with vehicle (control, open squares) or with an IL-17 inhibitor (SEQ ID NO: 1 (VHVTIPADLWDWINK)) formulated in phosphate-buffered saline at a concentration of 250 μM and administered twice daily for 7 days (triangles) or for 21 days (squares).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

“Amino acid(s)” refer to all naturally occurring L-amino acids and include D-amino acids. The amino acids are identified by either the single-letter or three-letter designations.

The term “amino acid sequence variant” refers to molecules with some differences in their amino acid sequences as compared to the sequences according to the present disclosure. Amino acid sequence variants of a polypeptide according to the present disclosure, e.g., of a specified sequence, retain the ability to bind the specific IL-17 cytokine or receptor, e.g., human IL-17A or, e.g., inhibit IL-17A binding to its receptor. Substitutional variants are those that have at least one amino acid residue removed and a different amino acid inserted in its place at the same position in a polypeptide according to the present disclosure, e.g., of a specified sequence. These substitutions may be single, where only one amino acid in the molecule has been substituted, or they may be multiple, where two or more amino acids have been substituted in the same molecule. Insertional variants are those with one or more amino acids inserted immediately adjacent to an amino acid at a particular position in a polypeptide, e.g., of a specified sequence. Immediately adjacent to an amino acid means connected to either the α-carboxy or α-amino functional group of the amino acid. Deletional variants are those with one or more amino acids in a polypeptide of a specified sequence removed.

“Identity” with respect to a native polypeptide and its functional derivative is defined as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are well known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The term “IL-17” refers to an IL-17 cytokine, such as IL-17A, IL-17B, IL-17C, IL-17D or IL-17E.

“IL-17A” intends the cytokine formerly known as CTLA8, and includes wild-type IL-17A from various species (e.g., human, mouse, and monkey), polymorphic variants of IL-17A, and functional equivalents of IL-17A. Functional equivalents of IL-17A have at least about 85%, 90%, 91%, 92%, 93%, 94%, 95%, 96%, 97%, 98%, or 99% overall sequence identity with a wild-type IL-17A (e.g., human IL-17A), and, in an embodiment, a functional fragment is one that substantially retains the ability to induce IL-6 production by human dermal fibroblasts.

An “IL-17 inhibitor” refers to a compound that binds an IL-17 cytokine and/or to an IL-17 receptor (e.g, IL-17RA, RB, RC, RD, or RE), to inhibit the interaction of the IL-17 cytokine with its receptor.

An “IL-17 cytokine inhibitor” refers to a compound that binds an IL-17 cytokine to inhibit its interaction with its receptor. In embodiments, the IL-17 cytokine inhibitor specifically binds a particular IL-17 cytokine, and is referred to according to the cytokine that it binds, for example, an “IL-17A cytokine inhibitor” refers to a compound that specifically binds IL-17A cytokine to inhibit its binding or its interaction with the IL-17A receptor, to thereby inhibit the formation of the IL-17A/IL-17RA/IL17RC complex.

A compound that “inhibits” one or more of the functional properties of IL-17 (e.g., biochemical, immunochemical, cellular, physiological or other biological activities, or the like) is determined according to methodologies known to the art and described herein, and achieves a statistically significant decrease in the particular activity relative to that seen in the absence of the compound (or when a control compound of irrelevant specificity is present). A compound that is an IL-17 inhibitor or an IL-17 cytokine inhibitor provides a statistically significant decrease, e.g., by at least 10% of the measured parameter, by at least 50%, 80% or 90%, and in certain embodiments may inhibit greater than 95%, 98% or 99% of IL-17 functional activity.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

As used herein, the term “subject” and “patient” includes any human or nonhuman animal. The term “nonhuman animal” includes all vertebrates, e.g., mammals and non-mammals, such as nonhuman primates, mice, rabbits, sheep, dogs, cats, horses, cows, chickens, amphibians, reptiles, etc.

The phrase “substantially identical” means that the relevant amino acid or nucleotide sequence will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region. In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity can be about 90% or greater, e.g., 90%, 91%, 92%, 93%, 94%, 95%, 96%, 97%, 98%, 99% or higher.

As used herein, a “therapeutically effective amount” refers to an amount of an IL-17 inhibitory that is effective, upon single or multiple dose administration to a subject (such as a human patient) to slow, inhibit, or delay progression of dry eye disease and/or to treat, resolve, or cure dry eye disease.

The terms “treatment” or “treat” refers to curative or disease modifying treatment, in patients who have been diagnosed as suffering from dry eye disease.

The compositions of the present disclosure can comprise, consist essentially of, or consist of, the components disclosed.

All percentages, parts and ratios are based upon the total weight of the compositions and all measurements made are at about 25° C., unless otherwise specified.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### II. Methods

Methods for treating dry eye disease and methods to slow, inhibit or delay progression of dry eye disease in a subject are provided. The methods comprise administering a composition comprising a therapeutically effective amount of a compound that inhibits binding of an inflammatory interleukin-17 (IL-17) cytokine to an IL-17 receptor, referred to as an IL-17 inhibitor. Studies conducted in support of the methods are described with respect to Examples 1-7.

In a first study, described in Example 1, treatment of dry eye disease with an IL-17 cytokine inhibitor, lifitegrast ophthalmic solution (XIIDRA®), and cyclosporine ophthalmic emulsion (RESTASIS®) were compared. The IL-17 cytokine inhibitor was an anti-IL-17A antibody formulated to 1 wt % in phosphate-buffered saline. Dry eye disease was induced in mice prior to treatment, and following induction of dry eye disease the mice were separated into groups for treatment with (i) the anti-IL-17A antibody once daily; (ii) 1 wt % anti-IL-17A antibody twice daily; (iii) 5 wt % lifitegrast twice daily; (iv) 0.05 wt % cyclosporine twice daily; or (v) saline twice daily as a control. The test compounds were applied topically to the eye for 9 days (from day 4 to day 12), and disease severity was evaluated using corneal fluorescein staining (CFS) and scored using the National Eye Institute Industry Workshop Scale of 0-15 on days 4, 7, 10 and 12.

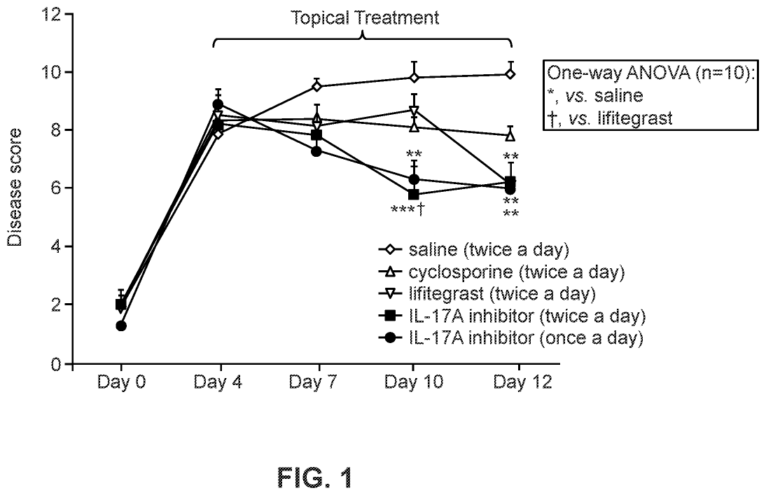

FIG. 1 is a graph of dry eye disease score as a function of time, in days, in subjects with dry eye disease treated by topical application to the eye with an anti-IL-17A antibody once daily (closed circles) or twice daily (closed squares), lifitegrast (XIIDRA®, 5 wt %) twice daily (inverted triangles), cyclosporine (RESTASIS®, 0.05 wt %) twice daily (triangles), or saline twice daily (diamonds). Topical treatment to the eye with the IL-17 cytokine inhibitor was more effective in treating dry eye disease, as measured by disease score, than lifitegrast or cyclosporine. Subject with dry eye disease scores of about 8 on day 4 of the study treated with cyclosporine had little, if any, reducing in disease severity, with the dry eye disease score remaining about 8 over the treatment period. Subject with dry eye disease scores of about 8 on day 4 of the study treated with lifitegrast showed no improvement in reducing in disease severity for the first six days of treatment, followed by an improved disease score at day 12. The subjects treated with the IL-17 cytokine inhibitor reported improved dry eye disease severity within 3 days of treatment, and a continued improvement in dry eye disease severity over the remainder of the treatment period.

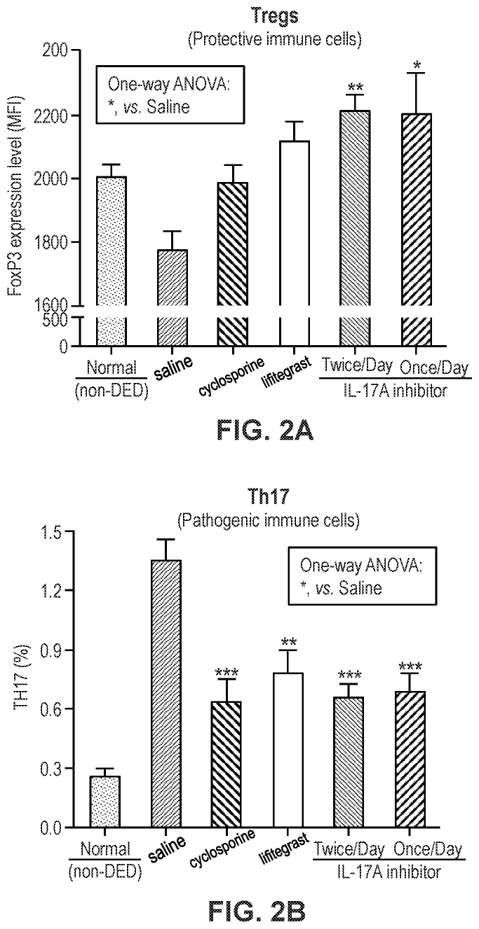

The data in FIG. 1 also shows that the IL-17 cytokine inhibitor was effective to treat dry eye disease when administered once per day. There was no statistical difference in the reduction in disease score in the test cohorts treated once daily or twice daily with the IL-17 cytokine inhibitor. This finding is also supported by the data shown in FIG. 2A, where treatment once per day or twice per day with the IL-17 cytokine inhibitor restored presence of protective immunoregulatory T cells (Tregs). FIG. 2B shows that treatment once per day or twice per day with the IL-17 cytokine inhibitor were both effective to decrease the percentage of pathogenic Th17 immune cells in the draining lymph nodes.

Accordingly, a method for treating dry eye disease is provided, where a subject with dry eye disease is treated with an IL-17 cytokine inhibitor. In one embodiment, the IL-17 cytokine inhibitor is administered to the eye once per day. In one embodiment, the IL-17 cytokine inhibitor is administered to the eye twice per day.

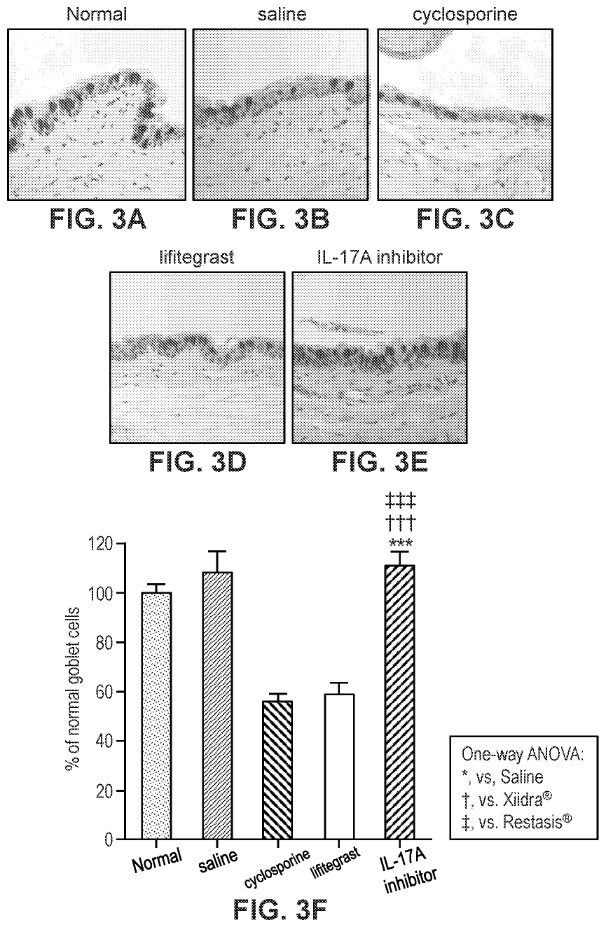

With continued reference to the study of Example 1, ocular tissue was harvested from the mice and tissue sections were prepared for microscopy analysis. Cross-sections of the conjunctiva were stained and the images were inspected to quantify goblet cell density. Goblet cells within the conjunctival epithelium secrete mucins onto the surface of the eye. FIG. 3A is an image of conjunctiva from a healthy mouse with no dry eye disease, and the goblet cells are visible as the darkly stained, plump cells. FIGS. 3B-3E are images from the conjunctiva from a subject in the animals with dry eye disease and treated twice daily with saline as a control (FIG. 3B), cyclosporine ophthalmic emulsion (RESTASIS®; FIG. 3C), lifitegrast ophthalmic solution (XIIDRA®) (FIG. 3D), or anti-IL-17A antibody (FIG. 3E). The numbers of goblet cells were counted, and FIG. 3F is a bar graph of the percent of normal goblet cells for the normal (healthy) mice and mice with dry eye disease treated with saline, cyclosporine, lifitegrast or an IL-17A inhibitor. Subjects treated with the IL-17A inhibitor had no loss in number of goblet cells, and the IL-17A inhibitor prevented goblet cell loss in the conjunctival tissue. Subjects with dry eye disease and treated with cyclosporine or with lifitegrast had about 60% of the normal number of goblet cells. Retention and/or restoration of goblet cell function and/or density is evidence that an IL-17A inhibitor is a disease-modifying therapy for dry eye disease, treating the disease itself rather than merely ameliorating the symptoms of the dry eye disease, and restores tear film homeostasis.

Dry eye disease is characterized by a reduction in tear volume, rapid breakup of the tear film, and/or an increase in the evaporative properties of the tear film layer. Tears are made of three layers, an outer oily or lipid layer, a middle watery layer, and an inner mucus layer. Production of a healthy tear film involves the meibomian glands, lacrimal glands, and goblet cells. The outer lipid layer of tear film is produced by the meibomian glands, and the layer functions to keep tear film on the surface of the eye from evaporating too quickly. The lacrimal glands produce the middle, aqueous layer, which comprises the bulk of tear volume and flow. This layer is an aqueous mixture that contains proteins, enzymes, antibodies and growth factors that are cytoprotective, anti-inflammatory, and anti-microbial. The aqueous layer nourishes the cornea and the conjunctiva that covers the entire front of the eye and the inside of the eyelids. The innermost mucin layer is produced by goblet cells and it functions to bind water from the aqueous layer to ensure that the eye remains wet. Dry eye disease is associated with a reduction in the function and/or density of conjunctival goblet cells, as seen by the data in FIGS. 3B and 3F. Accordingly, in one embodiment, a method to restore goblet cell function and/or density in an eye with signs or symptoms of dry eye disease is provided. In another embodiment, a method of preventing goblet cell loss in the eye in a person with dry eye disease is provided. The methods comprise topically administering to the eye of a subject in need, a therapeutically effective amount of a compound that inhibits an IL-17 cytokine from binding with an IL-17 receptor. In one embodiment, the compound inhibits IL-17A binding to IL-17A receptor.

In another embodiment, a method to improve balance of function of the lacrimal gland, the Meibomian glands and conjunctival goblet cells to restore tear film homeostasis in a subject is provided. The method comprises topically administering to the eye of a subject in need, a therapeutically effective amount of a compound that inhibits an IL-17 cytokine from binding with an IL-17 receptor. In one embodiment, the compound inhibits IL-17A binding to IL-17A receptor. In an embodiment, the subject in need is a person with dry eye disease.

In another embodiment, a method to increase basal tear production in elderly subjects is provided. The method comprises topically administering to the eye of a subject in need, a therapeutically effective amount of a compound that inhibits an IL-17 cytokine from binding with an IL-17 receptor. In one embodiment, the compound inhibits IL-17A binding to IL-17A receptor. In an embodiment, the subject in need is a person with dry eye disease. In one embodiment, the elderly subject has hormonal changes or is over age 50.

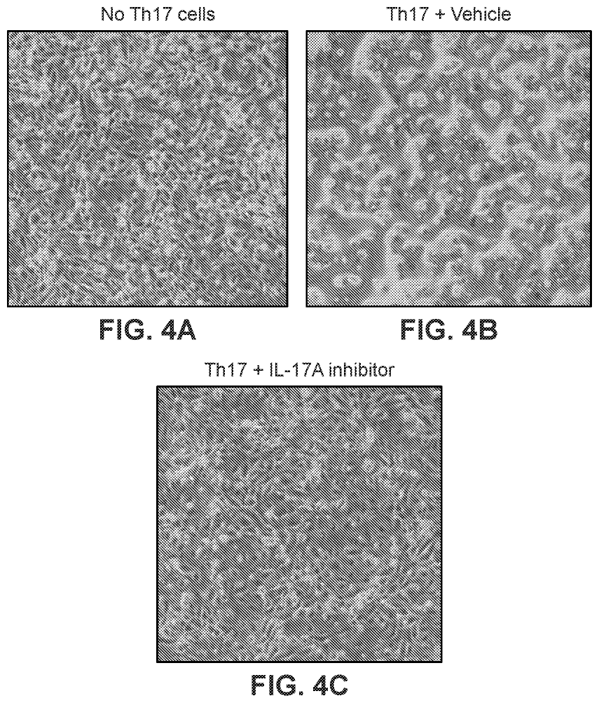

In another study, described in Example 2, a co-culture of human corneal epithelial cells and Th17 cells was prepared, to mimic the in vivo interaction between Th17 cells and cornea tissue in dry eye disease. The therapeutic efficacy of an IL-17 inhibitor, in the form of an antibody that inhibits IL-17A binding to its receptor, to protect the human corneal epithelial cells from Th17-induced damage was tested. The density and morphology of human corneal epithelial cells in the co-culture, with and without the IL-17 inhibitor, were observed under a bright-light inverted microscope equipped with a camera. Microscopic images of the cells are shown in FIGS. 4A-4C, where the image in FIG. 4A is of human corneal epithelial cells, with no Th17 cells or IL-17 treatment, as a control. FIG. 4B is an image of a co-culture of human corneal epithelial cells and Th17 cells treated with saline vehicle and FIG. 4C is an image of a co-culture of human corneal epithelial cells and Th17 cells treated with the IL-17 inhibitor. The IL-17 inhibitor protected the human corneal epithelial cells from Th17 mediated corneal damage, as apparent from the comparable cell density and morphology of the human corneal epithelial cells in the co-culture (FIG. 4C) and the control culture of human corneal epithelial cells (FIG. 4A).

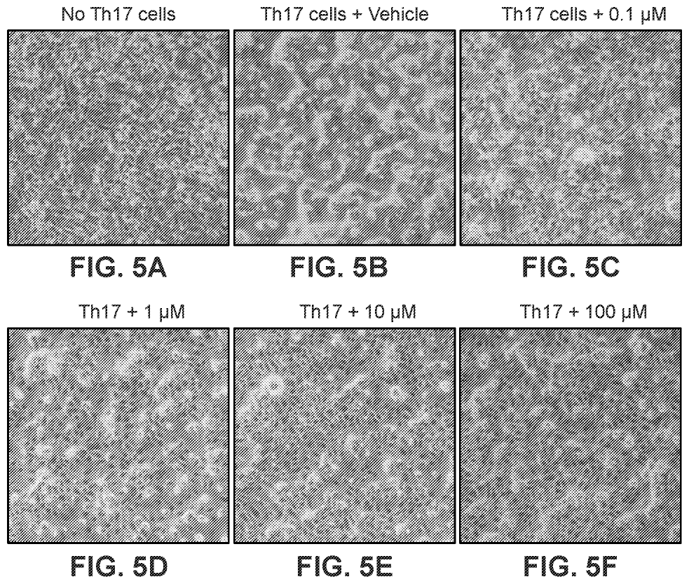

A similar study using the co-culture of human corneal epithelial cells and Th17 cells was done to test the therapeutic effect of an IL-17 inhibitor in the form of a peptide that inhibits IL-17A binding to the IL-17A receptor identified as SEQ ID NO: 1 (VHVTIPADLWDWINK), to assess whether the peptide protects the human corneal epithelial cells from Th17-induced damage. As described in Example 3, the IL-17 inhibitory peptide was formulated using phosphate buffered saline at appropriate concentrations to provide compositions with the IL-17 inhibitor at concentrations of 0.1 μM, 1 μM, 10 μM and 100 μM. After 24 hours of co-culture in the presence of the IL-17 inhibitor at each of the treatment doses, the density and morphology of human corneal epithelial cells were observed under a bright-light inverted microscope equipped with a camera. Representative images are shown in FIGS. 5A-5F. The image in FIG. 5A is of human corneal epithelial cells in culture, with no Th17 cells or IL-17 treatment, as a control. The image in FIG. 5B is of a co-culture of human corneal epithelial cells and Th17 cells treated with saline vehicle. The images in FIGS. 5C-5F are of co-cultures of human corneal epithelial cells and Th17 cells treated with the IL-17A inhibitor at concentrations of, respectively, 0.1 μM, 1 μM, 10 μM and 100 μM. After 24 hours, significant death of human corneal epithelial cells was observed in the cells treated only with vehicle (FIG. 5B). In contrast, the co-cultures treated with the IL-17 inhibitor at all concentrations protected the human corneal epithelial cells from Th17 mediated corneal damage (FIGS. 5C-5F), as apparent from the comparable cell density and morphology to the human corneal epithelial cell culture in the control (FIG. 5A). The images of the human corneal epithelial cells in FIGS. 5C-5F also shows that the IL-17 inhibitor was not toxic to the human corneal epithelial cells.

Example 4 describes a study that demonstrates the IL-17 inhibitor is effective to slow, inhibit and/or delay progression of dry eye disease. Four days after induction of dry eye disease, mice were treated with an IL-17 inhibitor. The IL-17 inhibitor was an anti-IL-17A antibody, at a dose of 1 wt % in phosphate-buffered saline, or a peptide that inhibits IL-17A binding to the IL-17A receptor, identified as SEQ ID NO: 1 (VHVTIPADLWDWINK). The IL-17A peptide inhibitor was formulated in phosphate-buffered saline at a dose of 0.05 wt % and 0.1 wt %. After nine days of treatment with the IL-17 inhibitory antibody or peptide, ocular surface damage was evaluated using corneal fluorescein staining (CFS) and scored using the National Eye Institute Industry Workshop Scale of 0-15 at day 0 (normal baseline), day 4 (before instillation of topical treatments), day 7, day 10 and day 12.

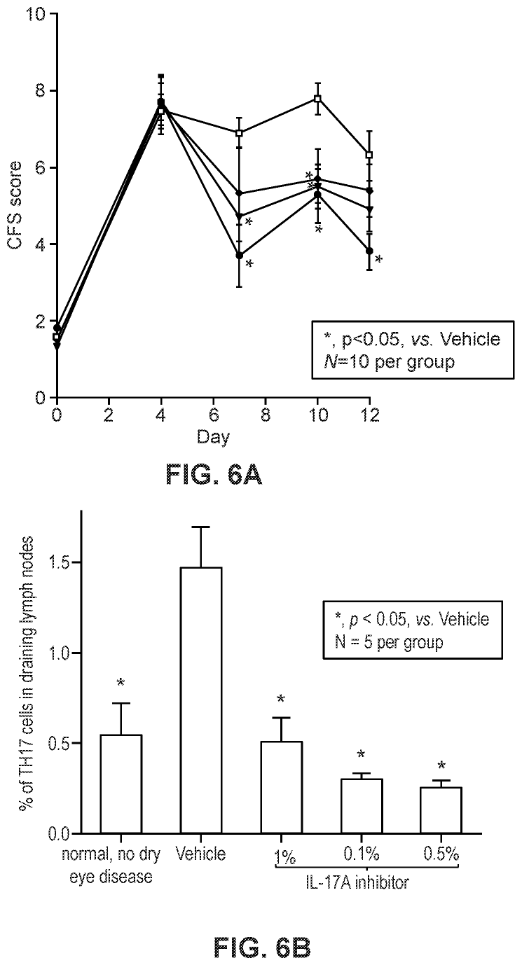

Results are shown in FIG. 6A, where the corneal fluorescein staining (CFS) scored using the National Eye Institute Industry Workshop Scale of 0-15 at day 0 (normal baseline) as a function of time is shown. The CFS score was determined on day 4 (before instillation of topical treatments) and during treatment with the IL-17 inhibitors. The data shows that both doses of the IL-17 inhibitory peptide (SEQ ID NO: 1, 0.05 wt % (circles) and 0.1 wt % (diamonds)) and the IL-17 inhibitory antibody (1 wt %, inverted triangles) effectively ameliorated the progression of dry eye disease compared to the vehicle treated cohort (open squares). In the absence of treatment with an IL-17 inhibitor, subjects not treated (open squares) had CFS scores approaching 8 on day 4 of the study, and the CFS score remained around 8 for the duration of the study, indicating continued progression of dry eye disease in the absence of treatment. In the subjects treated with an IL-17 inhibitor the CFS scores decreased upon treatment with the IL-17 inhibitors, with the formulations comprising an IL-17 inhibitor reducing severity of the dry eye disease and inhibiting its progression.

The eye-draining lymph nodes were collected at the end of treatment and the frequencies of IL-17+CD4+Th17 cells were determined in the tissue. Results are shown in FIG. 6B. The percent of Th17 cells in the draining lymph nodes of mice with no dry eye disease and of mice with dry eye disease and treated with saline vehicle (control) or with the IL-17 inhibitors is shown. The IL-17 inhibitors suppressed pathogenic Th17 cells in the ocular tissue.

Accordingly, in one embodiment, a method to slow, inhibit or delay progression of dry eye disease in a subject is provided. The method comprises administering directly to the eye of a subject in need a composition comprising a therapeutically effective amount of a compound that inhibits binding of an inflammatory interleukin-17 (IL-17) cytokine to an IL-17 receptor. In another embodiment, a method to reverse progression of dry eye disease in a subject is provided, where a compound that inhibits binding of an inflammatory interleukin-17 (IL-17) cytokine to an IL-17 receptor is administered directly to the eye of a subject in need thereof.

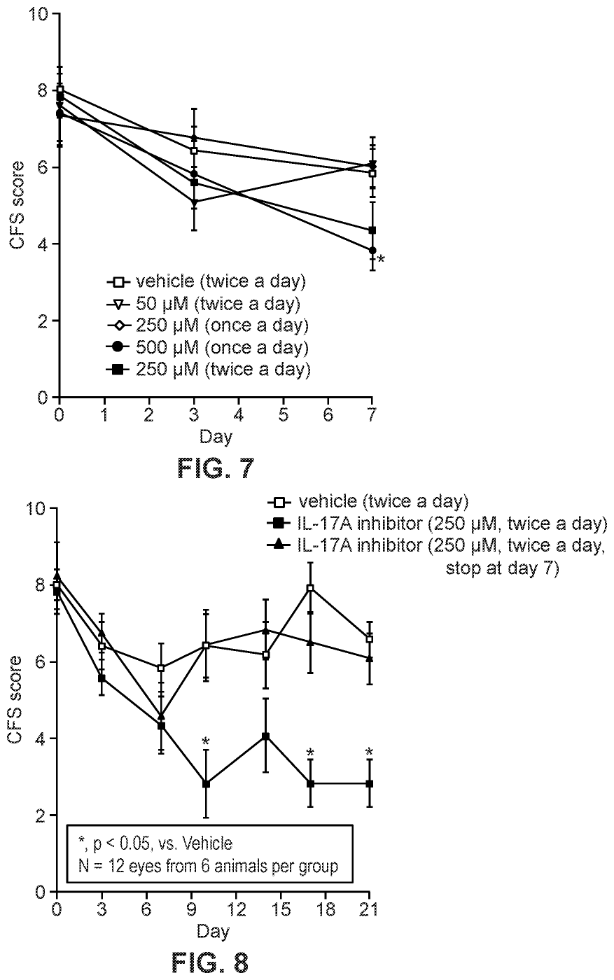

Additional studies were performed to further evaluate the IL-17A peptide inhibitor. Example 5 describes a study where mice with dry eye disease were treated with the IL-17A peptide inhibitor (SEQ ID NO: 1 (VHVTIPADLWDWINK)) at several dosages—50 μM, 250 μM and 500 μM, administered once or twice daily. FIG. 7 shows the results for the treatment groups for the first 7 days of the treatment period. The data in FIG. 7 shows that treatment of dry eye disease with the IL-17A peptide inhibitor at doses of 50 μM twice daily (inverted triangles) and at 250 μM administered once daily (diamonds) offered no or temporary improvement relative to treatment with saline twice daily (open squares). The subjects treated with the IL-17A inhibitor at doses of 250 μM administered twice daily (solid squares) and 500 μM administered once daily (circles) improved the disease, as seen by the marked decrease in CFS scores

In another study, dry eye disease was induced in mice as described in Example 5. Mice with similar dry eye disease severity were randomly divided into three treatment groups (n=6) for treatment after dry eye disease induction with the IL-17A peptide inhibitor (SEQ ID NO: 1) at a twice daily dose of 250 μM, where one group of mice were treated until day 7 of the study and vehicle thereafter, and another group of mice were treated with the IL-17 inhibitor for 21 days. The third group was a control group treated for 21 days with saline vehicle. All subjects in the cohorts were assessed at days 3, 7, 10, 14, 17 and 21 by corneal fluorescein staining (CFS) scored using the National Eye Institute Industry Workshop Scale of 0-15 and the results are shown in FIG. 8. Subjects in the vehicle (control) treatment group (squares) had continued progression and worsening of dry eye disease, as observed by the increase in CFS score over the 21 day study. Subjects treated with the IL-17 inhibitor experienced decreased CFS scores (squares, triangles), indicating improvement in the disease condition. Continued improvement in the disease severity, and inhibition of disease progression, was observed for the animals treated over the 21 day study (squares), whereas the subjects that were treated for seven days and then left untreated (triangles) showed return of disease severity and continued progression of the disease. Treatment of dry eye disease with the IL-17 inhibitor prevented and/or reduced progression of dry eye disease.

In an embodiment, a CFS score using the NEI/Industry Workshop criteria of between about 2-3 correlates to minimal dry eye disease, of between about 4-7 correlates to mild dry eye disease, of between about 8-10 correlates to moderate dry eye disease, and of between about 11-15 correlates to severe dry eye disease. Subjects in the study had an initial CFS score of around 8, or had moderate dry eye disease. In another embodiment, a CFS score using the VAS scale indicates minimal dry eye disease for a score between about 10-20, mild dry eye disease for a score between about 30-40, moderate dry eye disease for a score between about 50-70, and severe dry eye disease for a score between about 80-100. With reference to the study in Example 5 where the NEI/Industry Workshop scale for CFS scoring was used, treatment with the IL-17 inhibitor for 7 days improved the dry eye disease, with a CFS score at day 7 of 4.5-5. In the subjects that were treated with the IL-17 inhibitor to day 21, the CFS score continued to decrease, with a CFS score around 2.5 to 3 at days 18-21—which correlates to a treatment where the disease is minimal. However, cessation of treatment at day 7 resulted in the disease progressing from mild (CFS score of 4.5 at day 7) to the upper range of mild and approaching moderate (CFS score of 7 at day 14). Thus, the study demonstrates that treatment of dry eye disease with an IL-17 inhibitor is effective to prevent, delay, inhibit or reduce progression of the disease. In one embodiment, the treatment is effective to prevent, delay, inhibit or reduce progression of mild dry eye disease to moderate dry eye disease. In one embodiment, the treatment is effective to prevent, delay, inhibit or reduce progression of mild dry eye disease to severe dry eye disease. In one embodiment, the treatment is effective to prevent, delay, inhibit or reduce progression of moderate dry eye disease to severe dry eye disease.

The data in FIG. 8 also demonstrates that treatment of dry eye disease with the IL-17 inhibitor is a disease modifying therapy. Subjects with no dry eye disease have an average baseline CFS score of about 2. Treatment of subjects with dry eye disease with the IL-17 inhibitor, dosed daily, resolved the condition and achieved near disease resolution. The subjects with dry eye disease had an initial CFS score of around 8, and within 9 days of treatment with the IL-17 inhibitor the CFS score was approaching that of subjects with no dry eye disease. This result indicates that the method of treatment is a disease modifying therapy to achieve near or complete disease resolution. In one embodiment, the subject with dry eye disease is treated at least daily for a period of 2 weeks, 3 weeks, 4 weeks, 2 months, 3 months, 6 months or longer to substantially resolve the disease.

Also contemplated is a method to restore tear film homeostasis on the surface of an eye. Homeostasis refers to a condition of relatively stable equilibrium between interdependent elements, especially as maintained by physiological processes. With respect to the tear film, it is appreciated that the tear film composition is dynamic and in a constant state of flux, responding to environmental conditions in order to maintain ocular surface homeostasis. Traditionally, the tear film was described as being composed of three separate and distinct layers: mucin, aqueous, and lipid. However, it is now appreciated that mixing between the mucin and aqueous layer occurs, creating a gradient of decreasing mucin concentration into the aqueous layer. This aqueous-mucin layer forms a hydrated gel with complex biology, which is then covered by the lipid layer, which has its own highly ordered structure. Treatment with an IL-17 inhibitor restores tear film homeostasis such that the tear film in a person with dry eye disease is more like a person without dry eye disease. Measures of tear film homeostasis via tear film break up time, by way of an example, is described in Example 6. Tear film homeostasis can also be inferred from CFS scoring or from Schirmer's tests. Tear film break up time and ocular stating can also be used to assess the balance of function of the lacrimal gland, the Meibomian glands and conjunctival goblet cells to restore tear film homeostasis in a subject. A method to restore homeostasis of immunoregulatory function of regulatory T cells in the eye is also contemplated. Dry eye disease is, at least in part, an immunological response originating from an imbalance in the protective immunoregulatory versus Th17-mediated proinflammatory pathways of the ocular surface. Chronic dry eye disease involves a cycle of inflammatory responses perpetuated by the resistance of pathogenic, IL-17 cytokine secreting-Th17 cells to suppression by regulatory T cells (Treg). An unchecked Th17 activity perpetuates dry eye disease, while blocking IL-17 reduces inflammation at the ocular surface, to reduce the trafficking of immune cells from the ocular surface to the lymphoid tissue and to restore the immunoregulatory function of Tregs. Administration of an IL-17 inhibitor to the ocular region, and in particular to the ocular surface, reduces inflammation in the ye by blocking the pathogenic inflammatory cytokine of dry eye disease, IL-17. Blocking IL-17 leads to downregulation of inflammatory cytokines including TNF-α, IL-1, IL-6, IL-8, IL-23 and MMP-9. Blocking IL-17 also restores the immunoregulatory function of regulatory T cells (Treg), breaking the cycle of dry eye disease. Subsequently, other non-IL-17-associated contributors to inflammation are suppressed by Treg and the trafficking of immune cells from the ocular surface to lymphoid tissue is reduced.

### Composition Components

The methods described herein comprise administering to the eye a composition comprising a therapeutically effective amount of a compound that inhibits binding of an inflammatory interleukin-17 (IL-17) cytokine to an IL-17 receptor. Exemplary compounds and other composition components are now described.

IL-17 is a potent proinflammatory cytokine produced by a new lineage of CD4 T cells (Th17). IL-17 signals through a heteromeric receptor complex composed of IL-17RA and IL-17RC. IL-17 has pleiotropic effects on several immune and non-immune cells, providing an association between T cell activation and the inflammatory response. Furthermore, IL-17 cooperates either additively or synergistically with other proinflammatory cytokines such as TNFα, IL1β or IL6, leading to amplification of inflammatory processes. IL-17, also known as IL-17A, is part of a larger family comprising 6 cytokines, referred to as IL-17A, IL-17B, IL-17C, IL-17D, IL-17E, and IL-17F. All members of this family share a common protein structure. Among these family members, IL-17A and IL-17F are most frequently expressed in immune cells. In the methods described herein, one or more of these family members are targeted by antagonists to inhibit or modify their activity.

In one embodiment, the IL-17 inhibitor is a synthetic organic or inorganic compound. Macrocyclic compounds that inhibit interaction of IL-17 cytokine to its receptor include, for example, the macrocycles described in Wang et al., MedChemComm, 9(1):22-26 (2018) and sprirocycline indane compounds, such as those described in WO20200011731, incorporated by reference herein, or a spirocyclic oxoindoline, such as those described in WO2019/229079, incorporated by reference herein.

Embodiments wherein the compound that inhibits binding of an IL-17 cytokine to an IL-17 receptor is a protein or a peptide comprise administering a protein or a peptide that is purified and/or isolated. As used herein, an “isolated” or “purified” polypeptide or protein or peptide is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. Purity is measured by any appropriate standard method, for example, by column chromatography, polyacrylamide gel electrophoresis, or HPLC analysis.

In an embodiment, the IL-17 inhibitor is a biological compound such as an antibody or a peptide. Antibodies with specific binding for an IL-17 cytokine or an IL-17 receptor are described, for example, in U.S. Pat. No. 9,872,901 which is incorporated by reference herein. The antibody can be monoclonal or polyclonal. The contemplated antibody binds to one or more sequences within an IL-17 or IL-17 receptor polypeptide. In some embodiments, the antibody comprises a single chain antibody, a humanized antibody, a recombinant antibody, or a chimeric antibody. In other embodiments, the IL-17 inhibitor is an antibody fragment, including but not limited to Fab, F(ab′)₂, Fab′ Fv, single chain Fv, and the like. The antibody against IL-17 may be a reformulated or humanized derivative of or bind to the epitope of human IL-17 affinity purified polyclonal antibody, human IL-17 allophycocyanin monoclonal antibody, human IL-17 biotinylated affinity purified polyclonal antibody, human IL-17 monoclonal antibody, human IL-17 monoclonal antibody, human IL-17 phycoerythrin monoclonal antibody, mouse IL-17 affinity purified polyclonal antibody, mouse IL-17 biotinylated affinity purified polyclonal antibody, mouse IL-17 monoclonal antibody, or mouse IL-17 monoclonal antibody (each commercially available from, for example, R&D Systems). Preferably, the neutralizing or function-blocking antibody against IL-17 may be a reformulated or humanized derivative of or bind to the epitope of monoclonal anti-human IL-17 antibody, anti-human IL-17 antibody, polyclonal raised in goat, or recombinant human IL-17 R/Fc chimera. The antibody against an IL-17 receptor (IL-17R) may be a reformulated or humanized derivative of or bind to the epitope of human IL-17R affinity purified polyclonal antibody, human IL-17R allophycocyanin monoclonal antibody, human IL-17R biotinylated affinity purified polyclonal antibody, human IL-17R fluorescein monoclonal antibody, human IL-17R monoclonal antibody, human IL-17R monoclonal antibody, human IL-17R phycoerythrin monoclonal antibody, mouse IL-17R affinity purified polyclonal antibody, mouse IL-17R biotinylated affinity purified polyclonal antibody, or mouse IL-17R monoclonal antibody (each commercially available from R&D Systems).

In an embodiment, the IL-17 inhibitor is an antibody selected from secukinumab, ixekizumab, brodalumab, ABT-122, KHK 4827, perakizumab, RG 7624, ANB004 or COVA322. Secukinumab is a fully human IL-17A specific monoclonal antibody derived from human lgG1 kappa isotype and ixekizumab is a humanized lgG4 antibody. Secukinumab and ixekizumab are both specific for IL-17A homodimers and IL-17A. ABT-122 is a dual-variable-domain immunoglobulin targeting both TNF and IL-17A Brodalumab is a human mAb neutralizing IL-17RA with high affinity that can block the biological activity of IL-17, IL-17F, a heterodimer composed of 17A/17F, or 17E. In another embodiment, the antibody is as described in U.S. Pat. No. 9,765,140, incorporated by reference herein.

In one embodiment, the IL-17 inhibitor is a peptide that is able to inhibit IL-17A binding to IL-17RA. In one embodiment, the peptide binds with high affinity to IL-17A and inhibits its interaction with the ILRA receptor, to thereby inhibitor IL-17A signaling. Exemplary peptides are described in WO2020/021103, incorporated by reference herein. In one embodiment, the peptide comprises an amino acid sequence of Formula I.

X₁-X₂-X₃-X₄X₅-X₆-X₇-X₈-X₉-X₁₀-X₁₁-X₁₂-X₁₃-X₁₄-X₁₅ Formula (I),

wherein
X₁ is I, V or L;
X₂ is H, M, R, K or E;
X₃ is V, F or I;
X₄ is T, Q, S, Y or N;
X₅ is I, F or V;
X₆ is P or G;
X₇ is A, Q, or L;
X₈ is D, E, or Q;
X₉ is L, W, F, V or I;
X₁₀ is W, Y or F;
X₁₁ is D, E or N;
X₁₂ is W or F;
X₁₃ is I, V, F or L;
X₁₄ is N, R, Q or E;
X₁₅ is K, R, H or E;
   with the proviso that said sequence is not IHVTIPADLWDWINK (SEQ ID NO:167).

In another embodiment, Formula I is selected from
(a) X₁ is I or V,
   X₂ is H, M or R,
   X₃ is V or F;
   X₄ is T or Q;
   X₅ is I, F or V;
   X₆ is P or G;
   X₇ is A or Q;
   X₈ is D or E;
   X₉ is L;
   X₁₀ is W or Y;
   X is D or E;
   X₁₂ is W;
   X₁₃ is I or V;
   X₁₄ is N, R or E;
   X₁₅ is K, R or E;
with the proviso that said sequence is not IHVTIPADLWDWINK (SEQ ID NO: 167);
or
(b) X₁ is I or V;
   X₂ is H or M,
   X₃ is V;
   X₄ is T;
   X₅ is I;
   X₆ is P;
   X₇ is A;
   X₈ is D;
   X₉ is L, W, F, V or I;
   X₁₀ is W or Y;
   X₁₁ is D or E;
   X₁₂ is W;
   X₁₃ is I or V;
   X₁₄ is N, R or E;
   X₁₅ is K, R or E;
with the proviso that said sequence is not IHVTIPADLWDWINK (SEQ ID NO: 167), HVTIPADLWDWIN (SEQ ID NO: 168), IHVTIPADLWDWI (SEQ ID NO: 169) or IHVTIPADLWDW (SEQ ID NO: 170).

In an embodiment, the peptide is bound its C- and/or N-terminal to a protective cap group. Exemplary protective groups that can be bound to a C-terminal include amides, aldehydes, esters, p-nitroanilide, and 7-amino-4-methylcoumarin. Exemplary protection groups that can be bound to a N-terminal is selected from the group consisting of acetyl, formyl, pyroglutamyl, fatty acids, urea, carbamate sulfonamide, and alkylamine.

In another embodiment, one or more amino acid residues may be added at the N- or C-terminal of the peptides. In one embodiment, any of the peptides described herein can have an amino acid replacement that is conservative replacement, also referred to in the art as a conservative mutation or a conservative substitution. An amino acid replacement that changes a given amino acid to a different amino acid provides a mutated peptide with similar biochemical properties as the peptide without the conservative replacement.

In an embodiment, the peptide consists of a contiguous sequence of between 8-50, 8-40, 8-35, 8-30, 10-40, 10-35, 10-30, 10-25, 10-20 or 12-18 amino acid residues. In an embodiment, the peptide has at least about 70%, 75%, 80%, 85%, 90%, 91%, 92%, 93%, 94%, 95%, 95%, 97%, 98%, or 99% sequence identity to the peptides of Formula I. In another embodiment, the peptide has at least about 70%, 75%, 80%, 85%, 90%, 91%, 92%, 93%, 94%, 95%, 95%, 97%, 98%, or 99% sequence identity to the peptides of SEQ ID Nos: 1-166. In another embodiment, the peptide has at least about 70%, 75%, 80%, 85%, 90%, 91%, 92%, 93%, 94%, 95%, 95%, 97%, 98%, or 99% sequence identity to the peptides of SEQ ID Nos: 1-214.

The IL-17 inhibitor can also be a dimer of two such peptides or a bioconjugate comprised of an IL-17 inhibitory peptide attached to a biomolecule.

In an embodiment, the peptide in the form of a dimer formed by two peptides, wherein each peptide is a peptide of Formula I. In one embodiment, the two peptides in the dimer are linked by a polyethylene spacer.

In another embodiment, the peptide in the form of a bioconjugate comprising the peptide of Formula I and a biomolecule, wherein the biomolecule is bound to the N- and/or C-terminal of the peptide. The bioconjugate can optionally comprise a linker between the peptide and the biomolecule. Exemplary biomolecules include macromolecules, such as carbohydrates, lipids and proteins or small natural products. Other exemplary biomolecules are ascorbic acid, capric acid, capronic acid, N-acetyl-glucosamine (NAG), N-acetylmuramic acid (NAM), NAG-NAM, hyaluronic acid, alginic acid, chitin, (GalNAc)₂, Gal-alpha1,3-GalNAc and trigalacturonic acid.

The IL-17 inhibitor can be formulated into a composition for administration to the eye. In embodiments, the composition comprises saline or another ophthalmically acceptable buffer. The composition, in other embodiments, comprises one or more ophthalmically acceptable excipients, such as an alcohol (ethanol, propanol, and nonanol), a fatty alcohol (lauryl alcohol), a fatty acid (valeric acid, caproic acid and capric acid), a fatty acid ester (isopropyl myristate and isopropyl n-hexanoate), an alkyl ester (ethyl acetate and butyl acetate), a polyol (propylene glycol, propanedione and hexanetriol), a sulfoxide (dimethylsulfoxide and decylmethylsulfoxide), an amide (urea, dimethylacetamide and pyrrolidone derivatives), a surfactant (sodium lauryl sulfate, cetyltrimethylammonium bromide, polaxamers, spans, tweens, bile salts and lecithin), taerpene (d-limonene, alpha-terpeneol, 1,8-cineole and menthone), or an alkanone (N-heptane and N-nonane).

Optionally, the composition further contains a compound selected from the group consisting of a physiological acceptable salt, poloxamer analogs with carbopol, carbopol/hydroxypropyl methyl cellulose (HPMC), carbopol-methyl cellulose, carboxymethylcellulose (CMC), hyaluronic acid, cyclodextrin, and petroleum. Bioadhesive compositions are also contemplated, such as those comprising a polymer. Exemplary mucoadhesive polyanionic natural or semi-synthetic polymers include, but are not limited to, polygalacturonic acid, hyaluronic acid, carboxymethylamylose, carboxymethylchitin, chondroitin sulfate, heparin sulfate, and mesoglycan. Suitable hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon-caprolactone, polyanhydrides; polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers. In another embodiment, synthetic polymers and naturally-occurring polymers are contemplated, such as, but not limited to, collagen, fibrin, hyaluronic acid, agarose, and laminin-rich gels.

The composition is administered to the eye. In an embodiment, the composition is administered directly by topical application to the ocular surface. In another embodiment, the composition is administered directly by topical application to the conjunctival sac. In another embodiment, the composition is administered by instilling the composition into or onto the eye. In an embodiment, the composition is instilled as one or more drops, into the conjunctival sac or onto the ocular surface.

The dose of the IL-17 inhibitor will vary according to the compound, its potency and other factors. In an embodiment, the composition provides a therapeutically effective amount of the IL-17 inhibitor. By way of example, where the compound is an IL-17A peptide inhibitor, the therapeutically effective amount is typically at a total daily dose of between about 0.01-1,000 μM or between about 0.05-750 μM or between about 0.05-500 μM, or of at least about 500 μM or 1,000 μM.

### Dry Eye Questionnaires

In an embodiment, the methods comprise identifying a subject with dry eye disease and/or quantifying the severity of dry eye disease. In these embodiments, a questionnaire can be used to identify subjects with dry eye disease or to quantify severity of dry eye disease. In another embodiment, progression of dry eye disease, and more particularly, whether treatment delays, slows or inhibits progression of mild or moderate dry eye disease to moderate or severe dry eye disease, is assessed using a questionnaire.

An exemplary questionnaire is the Visual Analog Scale (VAS)-Symptom Index. The VAS questionnaire asks questions regarding ocular discomfort, and the subject is asked to subjectively rate each ocular symptom by placing a vertical mark on a horizontal line to indicate level of discomfort, where 0% corresponds to no discomfort and 100% corresponds to maximal discomfort. The ocular symptoms queried are burning/stinging, itching, foreign body sensation, eye discomfort, eye dryness, photophobia, and pain.

Another exemplary questionnaire is the Ocular Surface Disease Index (OSDI; Allergan Inc., Irvine, CA; Walt J. G. et al., Drug Inf J., 1997; 31:1436), which is a frequently used instrument to assess dry eye disease (Amparo, F., et al., Ophthalmology, 2015: 122(7):1498-1503). The questionnaire is comprised of 12 questions and evaluates the frequency of symptoms over the preceding week. The scores range from 0 to 100 and based on the score, the patients' symptoms can be categorized as normal (0-12), mild dry eye (13-22), moderate dry eye (23-32), or severe dry eye (33-100) (Schiffman R. M. et al., Arch Ophthalmol., 2000; 118:615-21; Miller K. L., Arch Ophthalmol., 2010; 128:94-101).

The Symptom Assessment Questionnaire iN Dry Eye (SANDE) is a questionnaire based on a visual analog scale that quantifies both severity and frequency of dry eye symptoms. The SANDE is comprised of two questions, and each question employs a 100 mm horizontal linear visual analog scale. The measurement of symptom frequency ranges from “rarely” to “all of the time,” and the symptom severity from “very mild” to “very severe.” (Gulati A. et al., Am J Ophthalmol., 2006; 142:125-31; Schaumberg D. A. et al., Ocul Surf, 2007; 5:50-7). Both tests are reliable and valid measures of dry eye symptoms (Schiffman R. M. et al., Arch Ophthalmol., 2000; 118:615-21; Gulati A. et al., Am J Ophthalmol., 2006; 142:125-31). The first question in the SANDE questionnaire probes frequency of symptoms by asking the subject to place an ‘x’ on a line ranging from “rarely” to “all the time” to indicate how often, on average, the persons' eyes feel dry and/or irritated. The second question assesses severity of symptoms by asking the asking the subject to place an ‘x’ on a line ranging from “very mild” to “very severe” to indicate how severe, on average, the person feels symptoms of dryness and/or irritation.

The Dry Eye Questionnaire (DEQ) diagnoses dry eye disease and quantifies its severity level. This questionnaire measures several symptoms using four variables: degree of irritation, frequency, intensity in the morning, and intensity late in the day (Chalmers R. L. et al., Contact Lens and Anterior Eye, 2010; 33(2):55-60). The DEQ allows for differentiating ophthalmic status: dry eye vs. non-dry eye patients, Sjögren syndrome and keratoconjunctivitis sicca vs. non-Sjögren syndrome and keratoconjunctivitis sicca patients, and control vs. Sjögren syndrome and keratoconjunctivitis sicca and non-Sjögren's syndrome and keratoconjunctivitis sicca patients (Begley C. G. et al., Cornea, 2002; 21(7):664-67.)

The SPEED questionnaire was designed by Korb and Blackie in order to quickly track the progression of dry eye symptoms over time (Blackie, C. et al., Ocular Surgery News, Europe Edition 2012). The SPEED questionnaire gives a score from 0 to 28 that is the result of assessing frequency and severity of symptoms. The symptoms assessed include dryness, grittiness, or scratchiness; soreness or irritation; burning or watering; and eye fatigue. The questionnaire measure frequency of each of the symptoms by asking if the symptoms occur never (0), sometimes (1), often (2) or constantly (3). Severity is assessed by asking whether these symptoms were not problematic (0), tolerable (1), uncomfortable (2), bothersome (3), or intolerable (4), where tolerable is defined as ‘not perfect, but not uncomfortable’; uncomfortable is “irritating, but does not interfere with my day’, bothersome is “irritating and interferes with my day” and intolerable is “unable to perform my daily tasks”.

Signs of dry eye disease can be measured in a variety of ways, including staining (e.g., corneal fluorescein staining and lissamine green conjunctival staining), analysis of biomarkers, tear film break-up time, Schirmer test (with or without anesthesia) or conjunctival hyperemia. Staining measures for assessment of dry eye disease include corneal staining with, for example, fluorescein or lissamine, and grading the staining using a system, such as the National Eye Institute (NEI) grading system or another scale. An exemplary NEI scale is the fluorescein staining scale that uses a standardized grading system of 0 to 3 for each of five areas on each cornea. A standard, exemplary NEI system uses a 0-15 point scale with fluorescein staining; another NEI system uses a 0-18 point scale and lissamine green conjunctival staining. The methods described herein may additionally comprise measuring, monitoring, or determining dry eye disease, and signs of dry eye disease using one or more of these measures. The methods described herein may additionally comprise identifying a subject with dry eye disease using one or more of these measures.

In one embodiment, the subject with dry eye disease is a subject that does not have dry eye disease due to Sjogren's syndrome, Meibomian gland dysfunction, uveitis, an intraocular condition or inflammation of interior tissues of the eye. In other embodiments, the IL-17 inhibitor nor the IL-17 cytokine inhibitor is not a peptide having a sequence identified herein as SEQ ID NO: 167-214.

### III. Examples

The following examples are illustrative in nature and are in no way intended to be limiting.

### Example 1

### Treatment of Dry Eye Disease

A study was conducted to compare treatment of dry eye disease with an IL-17 cytokine inhibitor, lifitegrast ophthalmic solution (XIIDRA®), and cyclosporine ophthalmic emulsion (RESTASIS®). The IL-17 cytokine inhibitor was an anti-IL-17A antibody (R&D Systems, Inc. Clone 50105) formulated to 1 wt % in phosphate-buffered saline. Six to eight-week old female C57BL/6 were obtained (Charles River Laboratories). Dry eye disease was induced by placing the mice in a controlled-environment chamber with a relative humidity of <20%, airflow of 15 L/min, and a constant temperature of 21-23° C. for 14 days. Corneal epithelial disease was evaluated using corneal fluorescein staining (CFS) and scored using the National Eye Institute Industry Workshop Scale of 0-15 at day 0 (normal baseline), day 4 (before instillation of topical treatments), day 7, day 10 and day 14.

Following induction of dry eye disease the mice were randomly assigned to a cohort for treatment with (i) the anti-IL-17A antibody once daily; (ii) the anti-IL-17A antibody twice daily; (iii) lifitegrast twice daily; (iv) cyclosporine twice daily; or (v) saline twice daily as a control. The test compounds were applied topically to the eye for 12 days, and each day disease severity was assessed with the National Eye Institute Industry Workshop Scale. Results are shown in FIG. 1.

Draining submandibular and cervical lymph nodes were harvested from the mice, and single-cell suspensions were prepared. The suspensions were analyzed as described in Chauhan, S. et al., J Immunology, 182:1247 (2009) for CD4+ CD25+ Foxp3+ Tregs and total CD4+ T cells. Results are shown in FIGS. 2A-2B.

Ocular tissue was also harvested from the mice and tissue sections were prepared for microscopy analysis. Cross-sectional images of conjunctival tissue are shown in FIGS. 3A-3E for healthy mice with no dry eye disease (FIG. 3A) and mice with dry eye disease treated twice daily with saline as a control (FIG. 3B), cyclosporine ophthalmic emulsion (RESTASIS®; FIG. 3C), lifitegrast ophthalmic solution (XIIDRA®) (FIG. 3D), or anti-IL-17A antibody (FIG. 3E). FIG. 3F is a bar graph of the percent of goblet cells for the normal (healthy) mice and mice with dry eye disease treated with saline, cyclosporine, liftegrast or an IL-17A inhibitor.

### Example 2

### Treatment of Dry Eye Disease

A co-culture of human corneal epithelial cells and Th17 cells was prepared, to mimic the in vivo interaction between Th17 cells and cornea tissue in dry eye disease. The therapeutic efficacy of an IL-17 inhibitor to protect the human corneal epithelial cells from Th17-induced damage was tested. Immortalized human corneal epithelial cells were obtained. To acquire human Th17 cells, naïve CD4+ T cells were first purified from primary human peripheral blood mononuclear cells (StemCell) by negative selection using MACS separation, follow by in vitro polarization to Th17 cells using human Th17 cell differentiation kit (RnD Systems) in X-VIVO 15 medium (Lonza). Successful differentiation of human Th17 cells was confirmed by flow cytometry before co-culture. At about 80% confluence of human corneal epithelial cell culture, 1×10⁵ Th17 cells were added, with and without an IL-17 inhibitor. After 24 hours of co-culture in the presence of the IL-17 inhibitor, the density and morphology of human corneal epithelial cells were observed under a bright-light inverted microscope equipped with a camera. Representative images are shown in FIGS. 4A-4C.

### Example 3

### Treatment of Dry Eye Disease

A co-culture of human corneal epithelial cells and Th17 cells was prepared, to mimic the in vivo interaction between Th17 cells and cornea tissue in dry eye disease. The therapeutic efficacy of an IL-17 inhibitor to protect the human corneal epithelial cells from Th17-induced damage was tested. Immortalized human corneal epithelial cells were obtained. To acquire human Th17 cells, naïve CD4+ T cells were first purified from primary human peripheral blood mononuclear cells (StemCell) by negative selection using MACS separation, follow by in vitro polarization to Th17 cells using human Th17 cell differentiation kit (RnD Systems) in X-VIVO 15 medium (Lonza). Successful differentiation of human Th17 cells was confirmed by flow cytometry before co-culture. At about 80% confluence of human corneal epithelial cell culture, 1×10⁵ Th17 cells were added, with and without an IL-17 inhibitor. The IL-17 inhibitor was a 15 amino acid peptide that inhibits IL-17A binding to the IL-17A receptor identified as SEQ ID NO: 1 (VHVTIPADLWDWINK). The IL-17 inhibitory peptide was formulated using phosphate buffered saline at appropriate concentrates to provide compositions with the IL-17 inhibitor at concentrations of 0.1 μM, 1 μM, 10 μM and 100 μM.

After 24 hours of co-culture in the presence of the IL-17 inhibitor at each of the treatment doses, the density and morphology of human corneal epithelial cells were observed under a bright-light inverted microscope equipped with a camera. Representative images are shown in FIGS. 5A-5F.

### Example 4

### Slowing Progression of Dry Eye Disease

Six to eight-week old female C57BL/6 mice were used in this study and dry eye disease was induced as described in Example 1. Four days after induction of dry eye disease the mice were randomized into four treatment cohorts (n=10) for treatment with saline or with an IL-17 inhibitor. Two IL-17 inhibitors were tested, an anti-IL-17A antibody (R&D Systems, Inc. Clone 50105) formulated to 1 w-t % in phosphate-buffered saline and a 15 amino acid peptide that inhibits IL-17A binding to the IL-17A receptor identified as SEQ ID NO: 1 (VHVTIPADLWDWINK) formulated in phosphate-buffered saline at doses of 0.05 wt % or 0.1 wt %. The treatment compositions, and the saline control, were administered topically by instillation into the eyes of 3 μL twice a day from days 4-12. Corneal epithelial disease was evaluated using corneal fluorescein staining (CFS) and scored using the National Eye Institute Industry Workshop Scale of 0-15 at day 0 (normal baseline), day 4 (before instillation of topical treatments), day 7, day 10 and day 12. Results are shown in FIG. 6A.

The eye-draining lymph nodes were collected at the end of treatment and the frequencies of IL-17+CD4+Th17 cells were analyzed by flow cytometry. The percent of Th17 cells in the draining lymph nodes for the treatment cohorts is shown in FIG. 6B.

### Example 5

### Slowing Progression of Dry Eye Disease

Six to eight-week old female C57BL/6 mice were used in this study and dry eye disease was induced as described in Example 1. Four days after induction of dry eye disease the mice were randomized into treatment cohorts (n=6) for treatment with saline or with an IL-17 inhibitor. The IL-17 inhibitor was a 15 amino acid peptide that inhibits IL-17A binding to the IL-17A receptor identified as SEQ ID NO: 1 (VHVTIPADLWDWINK) formulated in phosphate-buffered saline at concentrations of 50 μM, 250 μM and 500 μM. The formulation with 250 μM IL-17A inhibitor was administered to one treatment cohort once per day and to a second treatment cohort twice per day. The formulation with 50 μM IL-17A inhibitor was administered to a treatment cohort twice per day, and the formulation with 500 μM was administered to a cohort once daily. The treatment compositions, and the saline control, were administered topically by instillation of 3 μL into both eyes of the subjects. Table 5-1 summarizes the treatment cohorts and study design.

TABLE 5-1

| | | Dry Eye Disease | | | | | |
|---|---|---|---|---|---|---|---|
| | | Induction | | Dry Eye Disease Progression | | | |
| | | Induction Phase | | Treatment Phase | | | |
| Cohort | Treatment | Day −4 | Day 0 | Day 7 | Day 14 | Day 21 | Readouts |
| 1 | 250 μM | | | BID | BID | BID | CFS at D 0, D 3, D 7, |
| | (0.05 wt %) | | | | | | D 10, D 14, D 17, |
| 2 | 250 μM | | | BID | vehicle BID | vehicle BID | D 21 |
| | (0.05 wt %) | | | | | | |
| 3 | 50 μM | | | BID | Terminated | | |
| | (0.01 wt %) | | | | | | |
| 4 | 500 μM | | | QD + vehicle QD | Terminated | | |
| | (0.1 wt %) | | | | | | |
| 5 | 250 μM | | | QD + vehicle QD | Terminated | | |
| | (0.05 wt %) | | | | | | |
| 6 | vehicle | | | BID | BID | BID | |

Corneal epithelial disease was evaluated using corneal fluorescein staining (CFS) and scored using the National Eye Institute Industry Workshop Scale of 0-15 at day 0 (normal baseline), day 3 (before instillation of topical treatments), day 7, and day 10. The cohorts treated with the formulation with 250 μM IL-17A inhibitor were treated for 21 days, with CFS and scoring performed on days 3, 7, 10, 14, 17 and 21. Results are shown in FIG. 7 and FIG. 8.

### Example 6

### Restoration of Tear Film Homeostasis

Human subjects with moderate to severe dry eye disease are enrolled in a study for treatment with an IL-17 inhibitor. The IL-17 inhibitor is administered directly to the eyes of the subjects daily for 1 month. Prior to first dose, and then weekly thereafter, ocular surface assessments are performed.

Ocular surface staining using fluorescein and lissamine green will be assessed and recorded in the schematic representation of 5 corneal and 6 conjunctival regions per eye on the case report form using the National Eye Institute Industry Workshop Scales. A pictorial and descriptive grading scale (grades 0 to 15 or 0 to 18) are included on the case report form.
1. Corneal staining is assessed using 1.0 mg sodium fluorescein strips.
2. After wetting the end of the strip with a single drop of buffered saline, the excess is shaken into a waste bin with a sharp flick.
3. The lower lid is then pulled down and the flat end of the tip should be gently applied to the inferior tarsal conjunctiva with the intent of instilling a very small volume of dye and not inducing reflex tearing.
4. The patient will be instructed to blink naturally several times without forced closure of the eyelid to distribute the fluorescein.
5. After allowing fluorescein to remain on the eye for at least one minute, the 5 corneal regions will be graded using a yellow (Wratten #12) barrier filter in conjunction with the cobalt (blue) filter to maximize the view of the fluorescence. The upper eyelid is lifted slightly to grade the entire corneal surface. To enhance the contrast, position the yellow barrier filter in the path of the returning light (not in the path of the incident light).

Tear Film Breakup Time (TFBUT) will be assessed using slit lamp biomicroscopy according to the following steps.
1. The slit-lamp will be set to a magnification of approximately 10.times.
2. With adequate fluorescein in place (preferably using DET strips), the subject will be asked to stare straight ahead without blinking until told otherwise. The test should be performed in a room with no direct air on the patient's face.
3. A stopwatch will be used to record the time between the last complete blink and the first appearance of a growing micelle indicating tear-film breakup. If the patient blinks prematurely prior to the development of the breakup of the mires, the examiner should continue to try to obtain a reading.
4. Once TFBUT is observed, instruct patient to blink freely. This test should then be repeated a second time on the same eye.
5. If the difference between the first and second readings differs by more than two seconds, a third measurement should be performed and recorded.
6. This procedure will then be performed in the other eye.
7. It is recommended that TFBUT be performed in a room with a temperature of approximately 18° C. with a humidity of approximately 50%.

Ocular surface staining assessment will be completed with lissamine green conjunctival staining.
1. The lissamine green ophthalmic strip should be wetted with buffered saline and applied to the inferior tarsal conjunctiva. Care should be taken to instill adequate dye.
2. After allowing lissamine green to remain on the eye for one minute, the six nasal and temporal conjunctival regions will be graded.
3. To grade the temporal zone, the subject should be instructed to look nasally; to grade the nasal zone, the subject should be instructed to look temporally.
4. This procedure should then be completed in the other eye.

The study shows that chronic treatment with the IL-17 inhibitor improves TFBUT indicating restoration of tear film homeostasis.

### Example 7

### Method for Treating Dry Eye Disease

Human subjects with moderate to severe dry eye disease are enrolled in a study for treatment with an IL-17 inhibitor. The IL-17 inhibitor is a sprirocycline indane compound and is administered directly to the eyes of the subjects daily for 1 month. Prior to first dose, and then weekly thereafter, ocular surface and tear film assessments are performed as described in Example 6.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method to treat dry eye disease in a subject diagnosed with dry eye disease, comprising:
administering directly to the eye of a subject in need a composition comprising a therapeutically effective amount of a compound that inhibits binding of an inflammatory interleukin-17 (IL-17) cytokine to an IL-17 receptor.

2. A method to slow, inhibit or delay progression of dry eye disease in a subject, comprising:
administering directly to the eye of a subject in need a composition comprising a therapeutically effective amount of a compound that inhibits binding of an inflammatory interleukin-17 (IL-17) cytokine to an IL-17 receptor.

3. A method to reverse progression of dry eye disease in a subject, comprising:
administering directly to the eye of a subject in need a composition comprising a therapeutically effective amount of a compound that inhibits binding of an inflammatory interleukin-17 (IL-17) cytokine to an IL-17 receptor.

4. A method to restore tear film homeostasis on the surface of an eye, comprising:
administering directly to the eye of a subject in need a composition comprising a therapeutically effective amount of a compound that inhibits binding of an inflammatory interleukin-17 (IL-17) cytokine to an IL-17 receptor.

5. The method of claim 1, further comprising identifying a subject with mild or moderate dry eye disease.

6. The method of claim 1, further comprising identifying a subject with mild, moderate or severe dry eye disease.

7. The method of claim 5, wherein said identifying comprises use of a questionnaire.

8. The method of claim 7, wherein the questionnaire is selected from the Visual Analog Scale (VAS), the Ocular Surface Disease Index (OSDI) questionnaire, the Symptom Assessment Questionnaire iN Dry Eye (SANDE) questionnaire, Dry Eye Questionnaire (DEQ), and the Standard Patient Evaluation of Eye Dryness Questionnaire (SPEED).

9. The method of any one of claim 2, further comprising identifying a subject with mild or moderate dry eye disease and monitoring progression of dry eye disease using the Standard Patient Evaluation of Eye Dryness Questionnaire (SPEED).

10. The method of claim 9, wherein identifying comprises use of a questionnaire.

11. The method of claim 1, wherein said administering comprises administering a composition comprising a compound selected from a biological molecule or an organic synthetic compound.

12. The method of claim 11, wherein the organic, synthetic compound is a spirocyclic indane compound or a spirocyclic oxoindoline compound.

13. The method of claim 11, wherein the biological compound is an antibody with binding affinity for IL-17 cytokine or an antibody with binding affinity for an IL-17 receptor.

14. The method of claim 13, wherein the antibody is a monoclonal antibody, a polyclonal antibody, a single-chain antibody, a humanized, recombinant antibody, a chimeric antibody, or an antibody fragment.

15. The method of claim 13, wherein the antibody is selected from the group consisting of afasevikumab, bimekizumab, brodalumab, ixekizumab, izokibep netakimab, perakizumab, secukinumab, sonelokimab, tibulizumab, vunakizumab, ABY-035, CJM-112, CNTO-6785, DC-806/S-011806, FPP-003, GR-1501, HB-0017, IMU-035, LZM-012, QX-002N, BH-1657, HB-0043, HT-0017, ILCT-1001, IQ-001, LEO 153339/LP0200, LP-0200, MT-6194, MYMD-1, ND-016, SCT-650A, SM-17, YBL-004, ABM-60, ETI-1023, LQ-025, LQ-026, ABBV-257, AFB-035, ANB-004, BCD-121, COVA-322, CYT-017-IL17Qb, DLX-2882, DLC-2907, DLX-2909, DLX-3003, E-34935, E-35018, E-35762, E-36041, EBI-006, HEISCO-III-002, IL-17-RC, MEDI-571, MOR-106, MP-0230, PRS-190, SCH-900117, Y-320, ABT-122, BITS-7201A, CDP-435, EBI-028, IL-17E, JNJ-6118104, KHK-4827, and RG 7624.

16. The method of claim 11, wherein the biological compound is a protein or a peptide that specifically inhibits IL-17A binding to interleukin 17A receptor.

17. The method of claim 16, wherein the peptide consists of a contiguous sequence of between 12-18 amino acid residues, the contiguous sequence having at least about 70% sequence identity to SEQ ID NO: 1.

18. The method of claim 16, wherein the peptide comprises an amino acid sequence of Formula I:
X₁-X₂-X₃-X₄X₅-X₆-X₇-X₈-X₉-X₁₀-X₁I-X₁₂-X₁₃-X₁₄-X₁₅ Formula (I),
wherein
X₁ is I, V or L;
X₂ is H, M, R, K or E;
X₃ is V, F or I;
X₄ is T, Q, S, Y or N;
X₅ is I, F or V;
X₆ is P or G;
X₇ is A, Q, or L;
X₈ is D, E, or Q;
X₉ is L, W, F, V or I;
X₁₀ is W, Y or F;
X₁₁ is D, E or N;
X₁₂ is W or F;
X₁₃ is I, V, F or L;
X₁₄ is N, R, Q or E; and
X₁₅ is K, R, H or E.

19. The method of claim 18, wherein
(a) X₁ is I or V,
X₂ is H, M or R,
X₃ is V or F;
X₄ is T or Q;
X₅ is I, F or V;
X₆ is P or G;
X₇ is A or Q;
X₈ is D or E;
X₉ is L;
X₁₀ is W or Y;
X is D or E;
X₁₂ is W;
X₁₃ is I or V;
X₁₄ is N, R or E; and
X₁₅ is K, R or E;
or
(b) X₁ is I or V;
X₂ is H or M,
X₃ is V;
X₄ is T;
X₅ is I;
X₆ is P;
X₇ is A;
X₈ is D;
X₉ is L, W, F, V or I;
X₁₀ is W or Y;
X₁₁ is D or E;
X₁₂ is W;
X₁₃ is I or V;
X₁₄ is N, R or E; and
X₁₅ is K, R or E.

20. The method of claim 17, wherein the administering comprises administering the peptide bound its C- and/or N-terminal to a protective cap group, wherein the protective cap group bound to a C-terminal being is selected from the group consisting of amides, aldehydes, esters, p-nitroanilide, 7-amino-4-methylcoumarin and the protective group cap bound to a N-terminal is selected from the group consisting of acetyl, formyl, pyroglutamyl, fatty acids, urea, carbamate sulfonamide, and alkylamine.

21-35. (canceled)


SEQUENCE LISTING (214 sequences)

SEQ ID NO: 1 - protein, 15 aa, synthetic construct
VHVTIPADLWDWINK

SEQ ID NO: 2 - protein, 15 aa, synthetic construct
IMVTIPADLWDWINK

SEQ ID NO: 3 - protein, 15 aa, synthetic construct
VMVTIPADLWDWINK

SEQ ID NO: 4 - protein, 15 aa, synthetic construct
IHVTIPAELWDWINK

SEQ ID NO: 5 - protein, 15 aa, synthetic construct
IHVTIPADLYDWINK

SEQ ID NO: 6 - protein, 15 aa, synthetic construct
IHVTIPADLYEWINK

SEQ ID NO: 7 - protein, 15 aa, synthetic construct
IHVTIPADLWDWVNK

SEQ ID NO: 8 - protein, 15 aa, synthetic construct
IHVTIPADLWDWIRK

SEQ ID NO: 9 - protein, 15 aa, synthetic construct
IHVTIPADLWDWINR

SEQ ID NO: 10 - protein, 15 aa, synthetic construct
IHVTIPADLWDWIEK

SEQ ID NO: 11 - protein, 15 aa, synthetic construct
IHVTIPADLWDWINE

SEQ ID NO: 12 - protein, 15 aa, synthetic construct
IHVTIPADLYEWINK

SEQ ID NO: 13 - protein, 15 aa, synthetic construct
IHVTIPADLWDWVRR

SEQ ID NO: 14 - protein, 15 aa, synthetic construct
IHVTIPADLWDWVEE

SEQ ID NO: 15 - protein, 15 aa, synthetic construct
VMVTIPADLYEWINK

SEQ ID NO: 16 - skipped

SEQ ID NO: 17 - protein, 15 aa, synthetic construct
VMVTIPADLYEWIRR

SEQ ID NO: 18 - protein, 15 aa, synthetic construct
VMVTIPADLYEWIEE

SEQ ID NO: 19 - protein, 15 aa, synthetic construct
IMVTIPADLYEWIEE

SEQ ID NO: 20 - protein, 15 aa, synthetic construct
VHVTIPAELWEWVRR

SEQ ID NO: 21 - protein, 15 aa, synthetic construct
VHFTIPADLWEWVRR

SEQ ID NO: 22 - protein, 15 aa, synthetic construct
VHVQIPADLWEWVRR

SEQ ID NO: 23 - protein, 15 aa, synthetic construct
VHVTFPADLWEWVRR

SEQ ID NO: 24 - protein, 15 aa, synthetic construct
VHVTIPQDLWEWVRR

SEQ ID NO: 25 - protein, 15 aa, synthetic construct
VHVTIPANLWEWVRR

SEQ ID NO: 26 - protein, 15 aa, synthetic construct
VHVTIPADFWEWVRR

SEQ ID NO: 27 - protein, 15 aa, synthetic construct
VHVTIPADLYEWVRR

SEQ ID NO: 28 - protein, 15 aa, synthetic construct
VHVTIPADLWNWVRR

SEQ ID NO: 29 - protein, 15 aa, synthetic construct
VHVTIPADLWEFVRR

SEQ ID NO: 30 - protein, 15 aa, synthetic construct
VHVTIPADLWEWFRR

SEQ ID NO: 31 - protein, 15 aa, synthetic construct
VHVYIPAELWEWVRR

SEQ ID NO: 32 - protein, 15 aa, synthetic construct
VHVTIPAEWWEWVRR

SEQ ID NO: 33 - protein, 15 aa, synthetic construct
VHFTFPQDLWEWVRR

SEQ ID NO: 34 - protein, 15 aa, synthetic construct
VHFTFPQDFWEWVRR

SEQ ID NO: 35 - protein, 15 aa, synthetic construct
VHFTIPQDLYEWVRR

SEQ ID NO: 36 - protein, 15 aa, synthetic construct
VHFTFPQDLWNWVRR

SEQ ID NO: 37 - protein, 15 aa, synthetic construct
VHFTFPQDLWEFVRR

SEQ ID NO: 38 - protein, 15 aa, synthetic construct
VHFTFPQDLWEWFRR

SEQ ID NO: 39 - protein, 15 aa, synthetic construct
VHFQFPADLWEWVRR

SEQ ID NO: 40 - protein, 15 aa, synthetic construct
VHFQFPADFWEWVRR

SEQ ID NO: 41 - protein, 15 aa, synthetic construct
VHFQFPADLYEWVRR

SEQ ID NO: 42 - protein, 15 aa, synthetic construct
VHFQFPADLWNWVRR

SEQ ID NO: 43 - protein, 15 aa, synthetic construct
VHFQFPADLWEFVRR

SEQ ID NO: 44 - protein, 15 aa, synthetic construct
VHFQFPADLWEWFRR

SEQ ID NO: 45 - protein, 15 aa, synthetic construct
VHFQFPQDWWEWVRR

SEQ ID NO: 46 - protein, 15 aa, synthetic construct
VHFQIPQDWWEWVRR

SEQ ID NO: 47 - protein, 15 aa, synthetic construct
VHFQFPQDWYEWVRR

SEQ ID NO: 48 - protein, 15 aa, synthetic construct
VHFQFPQDWWNWVRR

SEQ ID NO: 49 - protein, 15 aa, synthetic construct
VHFQFPQDLWEFVRR

SEQ ID NO: 50 - protein, 15 aa, synthetic construct
VHFQFPQDWWEWFRR

SEQ ID NO: 51 - protein, 15 aa, synthetic construct
VHFTIPADFWEWFRR

SEQ ID NO: 52 - protein, 15 aa, synthetic construct
VHVQIPADFWEWFRR

SEQ ID NO: 53 - protein, 15 aa, synthetic construct
VHVTFPADLWEWFRR

SEQ ID NO: 54 - protein, 15 aa, synthetic construct
VHVTIPQDFWEWFRR

SEQ ID NO: 55 - protein, 15 aa, synthetic construct
VHFTIPQDWWEWVRR

SEQ ID NO: 56 - protein, 15 aa, synthetic construct
VHFTFPQDLYNWVRR

SEQ ID NO: 57 - protein, 15 aa, synthetic construct
VHFTFPQDLYNFVRR

SEQ ID NO: 58 - protein, 15 aa, synthetic construct
VHVTIPADLYNFFRR

SEQ ID NO: 59 - protein, 15 aa, synthetic construct
VHFQFPQDLWEWVRR

SEQ ID NO: 60 - protein, 14 aa, synthetic construct
VHFTIPQDLYNWRR

SEQ ID NO: 61 - protein, 15 aa, synthetic construct
VHFTIPADLYNFVRR

SEQ ID NO: 62 - protein, 15 aa, synthetic construct
VHFQIPQDLYNFFRR

SEQ ID NO: 63 - protein, 15 aa, synthetic construct
VHFQFPQEWYNWFRR

SEQ ID NO: 64 - protein, 15 aa, synthetic construct
VRFQFGQEWYNFFRR

SEQ ID NO: 65 - protein, 15 aa, synthetic construct
AHVTIPADLWDWINK

SEQ ID NO: 66 - protein, 15 aa, synthetic construct
GHVTIPADLWDWINK

SEQ ID NO: 67 - protein, 15 aa, synthetic construct
LHVTIPADLWDWINK

SEQ ID NO: 68 - protein, 15 aa, synthetic construct
PHVTIPADLWDWINK

SEQ ID NO: 69 - protein, 15 aa, synthetic construct
IRVTIPADLWDWINK

SEQ ID NO: 70 - protein, 15 aa, synthetic construct
IKVTIPADLWDWINK

SEQ ID NO: 71 - protein, 15 aa, synthetic construct
IEVTIPADLWDWINK

SEQ ID NO: 72 - protein, 15 aa, synthetic construct
IQVTIPADLWDWINK

SEQ ID NO: 73 - protein, 15 aa, synthetic construct
IYVTIPADLWDWINK

SEQ ID NO: 74 - protein, 15 aa, synthetic construct
IHATIPADLWDWINK

SEQ ID NO: 75 - protein, 15 aa, synthetic construct
IHGTIPADLWDWINK

SEQ ID NO: 76 - protein, 15 aa, synthetic construct
IHLTIPADLWDWINK

SEQ ID NO: 77 - protein, 15 aa, synthetic construct
IHPTIPADLWDWINK

SEQ ID NO: 78 - protein, 15 aa, synthetic construct
IHITIPADLWDWINK

SEQ ID NO: 79 - protein, 15 aa, synthetic construct
IHYTIPADLWDWINK

SEQ ID NO: 80 - protein, 15 aa, synthetic construct
IHWTIPADLWDWINK

SEQ ID NO: 81 - protein, 15 aa, synthetic construct
IHFTIPADLWDWINK

SEQ ID NO: 82 - protein, 15 aa, synthetic construct
IHVSIPADLWDWINK

SEQ ID NO: 83 - protein, 15 aa, synthetic construct
IHVYIPADLWDWINK

SEQ ID NO: 84 - protein, 15 aa, synthetic construct
IHVNIPADLWDWINK

SEQ ID NO: 85 - protein, 15 aa, synthetic construct
IHVQIPADLWDWINK

SEQ ID NO: 86 - protein, 15 aa, synthetic construct
IHVTAPADLWDWINK

SEQ ID NO: 87 - protein, 15 aa, synthetic construct
IHVTGPADLWDWINK

SEQ ID NO: 88 - protein, 15 aa, synthetic construct
IHVTLPADLWDWINK

SEQ ID NO: 89 - protein, 15 aa, synthetic construct
IHVTPPADLWDWINK

SEQ ID NO: 90 - protein, 15 aa, synthetic construct
IHVTVPADLWDWINK

SEQ ID NO: 91 - protein, 15 aa, synthetic construct
IHVTFPADLWDWINK

SEQ ID NO: 92 - protein, 15 aa, synthetic construct
IHVTYPADLWDWINK

SEQ ID NO: 93 - protein, 15 aa, synthetic construct
IHVTIAADLWDWINK

SEQ ID NO: 94 - protein, 15 aa, synthetic construct
IHVTIGADLWDWINK

SEQ ID NO: 95 - protein, 15 aa, synthetic construct
IHVTILADLWDWINK

SEQ ID NO: 96 - protein, 15 aa, synthetic construct
IHVTIVADLWDWINK

SEQ ID NO: 97 - protein, 15 aa, synthetic construct
IHVTIIADLWDWINK

SEQ ID NO: 98 - protein, 15 aa, synthetic construct
IHVTINADLWDWINK

SEQ ID NO: 99 - protein, 15 aa, synthetic construct
IHVTIPGDLWDWINK

SEQ ID NO: 100 - protein, 15 aa, synthetic construct
IHVTIPLDLWDWINK

SEQ ID NO: 101 - protein, 15 aa, synthetic construct
IHVTIPPDLWDWINK

SEQ ID NO: 102 - protein, 15 aa, synthetic construct
IHVTIPVDLWDWINK

SEQ ID NO: 103 - protein, 15 aa, synthetic construct
IHVTIPIDLWDWINK

SEQ ID NO: 104 - protein, 15 aa, synthetic construct
IHVTIPNDLWDWINK

SEQ ID NO: 105 - protein, 15 aa, synthetic construct
IHVTIPQDLWDWINK

SEQ ID NO: 106 - protein, 15 aa, synthetic construct
IHVTIPAQLWDWINK

SEQ ID NO: 107 - protein, 15 aa, synthetic construct
IHVTIPAYLWDWINK

SEQ ID NO: 108 - protein, 15 aa, synthetic construct
IHVTIPADAWDWINK

SEQ ID NO: 109 - protein, 15 aa, synthetic construct
IHVTIPADGWDWINK

SEQ ID NO: 110 - protein, 15 aa, synthetic construct
IHVTIPADPWDWINK

SEQ ID NO: 111 - protein, 15 aa, synthetic construct
IHVTIPADVWDWINK

SEQ ID NO: 112 - protein, 15 aa, synthetic construct
IHVTIPADIWDWINK

SEQ ID NO: 113 - protein, 15 aa, synthetic construct
IHVTIPADLHDWINK

SEQ ID NO: 114 - protein, 15 aa, synthetic construct
IHVTIPADLFDWINK

SEQ ID NO: 115 - protein, 15 aa, synthetic construct
IHVTIPADLWNWINK

SEQ ID NO: 116 - protein, 15 aa, synthetic construct
IHVTIPADLWDHINK

SEQ ID NO: 117 - protein, 15 aa, synthetic construct
IHVTIPADLWDFINK

SEQ ID NO: 118 - protein, 15 aa, synthetic construct
IHVTIPADLWDYINK

SEQ ID NO: 119 - protein, 15 aa, synthetic construct
IHVTIPADLWDWENK

SEQ ID NO: 120 - protein, 15 aa, synthetic construct
IHVTIPADLWDWKNK

SEQ ID NO: 121 - protein, 15 aa, synthetic construct
IHVTIPADLWDWANK

SEQ ID NO: 122 - protein, 15 aa, synthetic construct
IHVTIPADLWDWGNK

SEQ ID NO: 123 - protein, 15 aa, synthetic construct
IHVTIPADLWDWLNK

SEQ ID NO: 124 - protein, 15 aa, synthetic construct
IHVTIPADLWDWPNK

SEQ ID NO: 125 - protein, 15 aa, synthetic construct
IHVTIPADLWDWFNK

SEQ ID NO: 126 - protein, 15 aa, synthetic construct
IHVTIPADLWDWYNK

SEQ ID NO: 127 - protein, 15 aa, synthetic construct
IHVTIPADLWDWIQK

SEQ ID NO: 128 - protein, 15 aa, synthetic construct
IHVTIPADLWDWIDK

SEQ ID NO: 129 - protein, 15 aa, synthetic construct
IHVTIPADLWDWINH

SEQ ID NO: 130 - protein, 15 aa, synthetic construct
IHVTIPADLWDWIND

SEQ ID NO: 131 - protein, 15 aa, synthetic construct
VHVTVPQELWEWVRR

SEQ ID NO: 132 - protein, 15 aa, synthetic construct
VHVTVPQELFEWVRR

SEQ ID NO: 133 - protein, 15 aa, synthetic construct
VHVTVPQELYEWVRR

SEQ ID NO: 134 - protein, 15 aa, synthetic construct
VHVTVPQELWEWVEE

SEQ ID NO: 135 - protein, 15 aa, synthetic construct
VHVTVPQELFEWVEE

SEQ ID NO: 136 - protein, 15 aa, synthetic construct
VHVTVPQELYEWVEE

SEQ ID NO: 137 - protein, 15 aa, synthetic construct
VHVSIPQELWEWVRR

SEQ ID NO: 138 - protein, 15 aa, synthetic construct
VHVSVPQELWEWVRR

SEQ ID NO: 139 - protein, 15 aa, synthetic construct
VHVSVPQELYEWVRR

SEQ ID NO: 140 - protein, 15 aa, synthetic construct
VRVTIPQELWEWVRR

SEQ ID NO: 141 - protein, 15 aa, synthetic construct
VRVTVPQELYEWVRR

SEQ ID NO: 142 - protein, 15 aa, synthetic construct
VRVTVPQELWEWVRR

SEQ ID NO: 143 - protein, 15 aa, synthetic construct
VHVTVPQEIYEWVRR

SEQ ID NO: 144 - protein, 15 aa, synthetic construct
VHVTIPQEIWEWVRR

SEQ ID NO: 145 - protein, 15 aa, synthetic construct
VHFTVPQELYEWVRR

SEQ ID NO: 146 - protein, 15 aa, synthetic construct
VKISVPADLWDWINK

SEQ ID NO: 147 - protein, 15 aa, synthetic construct
LRISVPADLWDWINK

SEQ ID NO: 148 - protein, 15 aa, synthetic construct
LRIYVPADLWDWINK

SEQ ID NO: 149 - protein, 15 aa, synthetic construct
VRGYVPADLWDWINK

SEQ ID NO: 150 - protein, 15 aa, synthetic construct
VRAYVPADLWDWINK

SEQ ID NO: 151 - protein, 15 aa, synthetic construct
VRLYVPADLWDWINK

SEQ ID NO: 152 - protein, 15 aa, synthetic construct
VRIYLPADLWDWINK

SEQ ID NO: 153 - protein, 15 aa, synthetic construct
IHVTIPLEIFEWLQH

SEQ ID NO: 154 - protein, 15 aa, synthetic construct
IHVTIPLEIFEWAQH

SEQ ID NO: 155 - protein, 15 aa, synthetic construct
IHVTIPLEIFEWLQR

SEQ ID NO: 156 - protein, 15 aa, synthetic construct
IHVTIPLEVFEWLQH

SEQ ID NO: 157 - protein, 15 aa, synthetic construct
IHVTIPGEIFEWLQH

SEQ ID NO: 158 - protein, 15 aa, synthetic construct
VRFSVPQEIYEWVRR

SEQ ID NO: 159 - protein, 15 aa, synthetic construct
LRISVPLEIFEWLQH

SEQ ID NO: 160 - protein, 15 aa, synthetic construct
LRGSVPLEIFEWLQH

SEQ ID NO: 161 - protein, 15 aa, synthetic construct
VKISVPLEIFEWLQH

SEQ ID NO: 162 - protein, 15 aa, synthetic construct
VEFNFPQQVYEWFDD

SEQ ID NO: 163 - protein, 15 aa, synthetic construct
VEFNFPQQVYEWVRR

SEQ ID NO: 164 - protein, 15 aa, synthetic construct
TWYVFNEQHQEYVRK

SEQ ID NO: 165 - protein, 15 aa, synthetic construct
VPGAGVPGAGIHVTI

SEQ ID NO: 166 - protein, 17 aa, synthetic construct
VPGAGVPGAGIHVTIPA

SEQ ID NO: 167 - protein, 15 aa, synthetic construct
IHVTIPADLWDWINK

SEQ ID NO: 168 - protein, 13 aa, synthetic construct
HVTIPADLWDWIN

SEQ ID NO: 169 - protein, 13 aa, synthetic construct
IHVTIPADLWDWI

SEQ ID NO: 170 - protein, 12 aa, synthetic construct
IHVTIPADLWDW

SEQ ID NO: 171 - protein, 16 aa, synthetic construct
DLSAVCWAFPWDPECH

SEQ ID NO: 172 - protein, 20 aa, synthetic construct
DSSAVCWAFPHHPLCHMKAT

SEQ ID NO: 173 - protein, 16 aa, synthetic construct
ADADMCWFFPTSPWCH

SEQ ID NO: 174 - protein, 16 aa, synthetic construct
DSSAVCWAFPYLPECH

SEQ ID NO: 175 - protein, 16 aa, synthetic construct
DISAVCWAFPFDPECH

SEQ ID NO: 176 - protein, 20 aa, synthetic construct
AYECPRLEYDMFGALHCLPS

SEQ ID NO: 177 - protein, 15 aa, synthetic construct
CPRLEYDMFGALHCL

SEQ ID NO: 178 - protein, 15 aa, synthetic construct
CLDLQYDPWGALHCI

SEQ ID NO: 179 - protein, 15 aa, synthetic construct
CFDLQYDPWGALHCI

SEQ ID NO: 180 - protein, 15 aa, synthetic construct
CLDLQYDMFGALHCV

SEQ ID NO: 181 - protein, 15 aa, synthetic construct
CLDLVYDPWGALHCI

SEQ ID NO: 182 - protein, 15 aa, synthetic construct
CWVLEYDMFGALHCR

SEQ ID NO: 183 - protein, 15 aa, synthetic construct
CWALEYDMFGYLHCR

SEQ ID NO: 184 - protein, 15 aa, synthetic construct
CWVLEYDMFGFLHCR

SEQ ID NO: 185 - protein, 15 aa, synthetic construct
CWVLEYDMFGYLHCR

SEQ ID NO: 186 - protein, 19 aa, synthetic construct
GPYYFDSSGYLYYYYGLDV

SEQ ID NO: 187 - protein, 15 aa, synthetic construct
IVVTMPADLWDWIKA

SEQ ID NO: 188 - protein, 15 aa, synthetic construct
IVVTMPADLWDWIRA

SEQ ID NO: 189 - protein, 15 aa, synthetic construct
IVVTMPADLWDWIRK

SEQ ID NO: 190 - protein, 15 aa, synthetic construct
IVVTMPADLWDWIAA

SEQ ID NO: 191 - protein, 15 aa, synthetic construct
IVVTMPADLWDWARA

SEQ ID NO: 192 - protein, 15 aa, synthetic construct
IVVTMPADLWDAIRA

SEQ ID NO: 193 - protein, 15 aa, synthetic construct
IVVTMPADLWAWIRA

SEQ ID NO: 194 - protein, 15 aa, synthetic construct
IVVTMPADLADWIRA

SEQ ID NO: 195 - protein, 15 aa, synthetic construct
IVVTMPADAWDWIRA

SEQ ID NO: 196 - protein, 15 aa, synthetic construct
IVVTMPAALWDWIRA

SEQ ID NO: 197 - protein, 15 aa, synthetic construct
IVVTMPADLWDWIRA

SEQ ID NO: 198 - protein, 15 aa, synthetic construct
IVVTMAADLWDWIRA

SEQ ID NO: 199 - protein, 15 aa, synthetic construct
IVVTAPADLWDWIRA

SEQ ID NO: 200 - protein, 15 aa, synthetic construct
IVVAMPADLWDWIRA

SEQ ID NO: 201 - protein, 15 aa, synthetic construct
IVATMPADLWDWIRA

SEQ ID NO: 202 - protein, 15 aa, synthetic construct
IAVTMPADLWDWIRA

SEQ ID NO: 203 - protein, 15 aa, synthetic construct
AVVTMPADLWDWIRA

SEQ ID NO: 204 - protein, 15 aa, synthetic construct
IHVTMPADLWDWIRA

SEQ ID NO: 205 - protein, 15 aa, synthetic construct
IQVTMPADLWDWIRA

SEQ ID NO: 206 - protein, 15 aa, synthetic construct
IRVTMPADLWDWIRA

SEQ ID NO: 207 - protein, 15 aa, synthetic construct
ITVTMPADLWDWIRA

SEQ ID NO: 208 - protein, 15 aa, synthetic construct
IWVTMPADLWDWIRA

SEQ ID NO: 209 - protein, 15 aa, synthetic construct
IYVTMPADLWDWIRA

SEQ ID NO: 210 - protein, 15 aa, synthetic construct
IVVTIPADLWDWIRA

SEQ ID NO: 211 - protein, 15 aa, synthetic construct
IVVTLPADLWDWIRA

SEQ ID NO: 212 - protein, 15 aa, synthetic construct
IVVTVPADLWDWIRA

SEQ ID NO: 213 - protein, 15 aa, synthetic construct
IVVTMPADLWDWIMA

SEQ ID NO: 214 - protein, 15 aa, synthetic construct
IVVTMPADLWDWINA